(19) United States
(12) Patent Application Publication
(10) Pub. No.: US 2025/0235440 A1
(43) Pub. Date: Jul. 24, 2025

(54) IMPLANTABLE DEPOTS WITH HIGH THERAPEUTIC PAYLOADS

(71) Applicant: Allay Therapeutics, Inc., Menlo Park, CA (US)

(72) Inventors: Daniel Boon Lim Seet, Singapore (SG); Jackie Joe Hancock, Albany, CA (US); Cynthia R. Lee, Belmont, CA (US); Ming Siew Lim, Singapore (SG); Alicia Mui Shen Ng, Singapore (SG); Patrick H. Ruane, El Dorado Hills, CA (US); Megan Wei Wei Soo, Singapore (SG); James Su, Newark, CA (US); Koon Kiat Teu, Singapore (SG)

(21) Appl. No.: 19/061,443

(22) Filed: Feb. 24, 2025

Related U.S. Application Data
(63) Continuation of application No. PCT/US23/72669, filed on Aug. 22, 2023.
(60) Provisional application No. 63/373,506, filed on Aug. 25, 2022.
(60) Provisional application No. 63/373,508, filed on Aug. 25, 2022.

(51) Int. Cl. A61K 31/445; A61K 9/00; A61L 31/16

(52) U.S. Cl. CPC A61K 31/445; A61K 9/0024; A61L 31/16; A61L 2300/402; A61L 2300/602; A61L 2300/606

(57) ABSTRACT

Implantable depots for the sustained, controlled release of therapeutic agents, and associated systems and methods, are disclosed herein. In some embodiments, for example, an implantable depot for treating pain includes an analgesic constituting at least 50% of a total mass of the implantable depot. At least some or all of the analgesic can be in a free base form. When implanted at a treatment site in vivo, the implantable depot can be configured to release the analgesic over a release period of at least 3 days.

### CROSS-REFERENCE TO RELATED APPLICATION(S)

This application is a continuation of International Patent Application No. PCT/US2023/072669, filed Aug. 22, 2023, which claims the benefit of priority to U.S. Provisional Application No. 63/373,506, filed Aug. 25, 2022, and U.S. Provisional Application No. 63/373,508, filed Aug. 25, 2022, each of which is incorporated by reference herein in its entirety.

### TECHNICAL FIELD

The present technology relates generally to implantable depots, and in particular, to implantable depots with high therapeutic payloads.

### BACKGROUND

Implantable systems for the controlled release of therapeutic agents offer advantages over other drug delivery methods, such as oral or parenteral methods. Devices made of biocompatible and/or biodegradable materials and therapeutic agents can be implanted in clinically desirable anatomic locations, thereby providing localized delivery of select agents. This localized delivery enables a substantial proportion of the agent to reach the intended target and undesirable systemic side effects can be avoided. However, these systems often suffer from a lack of a true controlled release mechanism in that they typically provide a burst release of therapeutic agent upon contact with surrounding physiologic fluids, followed by a residual release of agent.

Additionally, conventional implantable systems may require a relatively large proportion of non-therapeutic substances, such as carriers, fillers, excipients, etc. For example, many conventional drug delivery technologies rely on the properties of a carrier material to package and control the delivery of the therapeutic agent. These additional components may reduce the amount of therapeutic agent that can be incorporated into the implant, increase the size of the implant, increase manufacturing costs and complexity, and present regulatory challenges.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A illustrates the free base and hydrochloride salt forms of bupivacaine, in accordance with embodiments of the present technology.

FIG. 1B illustrates cellular uptake of an amine-containing local anesthetic, in accordance with embodiments of the present technology.

FIG. 1C is a perspective view of an implantable depot configured in accordance with embodiments of the present technology.

FIG. 1D is a top view of the implantable depot of FIG. 1C.

FIG. 2 is a top view of a triangular depot configured in accordance with embodiments of the present technology.

FIG. 3 is a top view of a circular depot configured in accordance with embodiments of the present technology.

FIG. 4 is a perspective view of a spherical depot configured in accordance with embodiments of the present technology.

FIG. 5 is a perspective view of a cylindrical or tubular depot configured in accordance with embodiments of the present technology.

FIG. 6 is a side cross-sectional view of an implantable depot in the form of a coating on a device, in accordance with embodiments of the present technology.

FIGS. 7A-7D are photographs illustrating a melt form process for manufacturing an implantable depot, in accordance with embodiments of the present technology.

FIGS. 7E and 7F are scanning electron microscopy images of depots formed using the melt form process, in accordance with embodiments of the present technology.

FIGS. 8A-8D are photographs illustrating melt-formed, remolded implantable depots with various geometries, in accordance with embodiments of the present technology.

FIGS. 9A-9C are photographs illustrating melt-formed implantable depots with various geometries, in accordance with embodiments of the present technology.

FIG. 9D is a graph illustrating cumulative in vitro release of bupivacaine free base from the implantable depots of FIGS. 9A-9C.

FIG. 10 is a semilog graph illustrating in vivo release of bupivacaine free base from an implantable depot in a rabbit subcutaneous model.

FIGS. 11A-11E are photographs illustrating a dip coating process for manufacturing coated implantable meshes, in accordance with embodiments of the present technology.

FIG. 11F is a graph illustrating cumulative in vitro release of bupivacaine free base from a coated mesh.

FIG. 11G is a scanning electron microscopy image of an uncoated mesh.

FIG. 11H is a scanning electron microscopy image of the mesh of FIG. 11G after coating.

FIGS. 11I and 11J are scanning electron microscopy images of a coated mesh at various time points during an in vitro release test.

FIG. 12 is a graph illustrating cumulative in vitro release of bupivacaine free base from a powder.

FIGS. 13A-13C are scanning electron microscopy images of bupivacaine free base powder with varying particle sizes.

FIG. 13D is a graph illustrating cumulative in vitro release of bupivacaine free base from samples with varying form factors.

### DETAILED DESCRIPTION

The present technology relates to implantable depots for the sustained, controlled release of therapeutic agents, and associated systems and methods. In some embodiments, for example, an implantable depot for treating pain includes an analgesic constituting a majority of the total mass of the implantable depot, such as at least 50%, 80%, 90%, or 100% of the total mass of the depot. At least some or all of the analgesic can be in a free base form. Optionally, some of the analgesic can be in a different form, such as a salt form. When implanted at a treatment site in vivo, the implantable depot can be configured to release the analgesic over a release period of at least 3 days.

The implantable depots described herein can provide numerous advantages compared to conventional drug delivery systems. For example, conventional drug delivery systems typically require a carrier material (e.g., polymers, liposomes, lipids, micelles, coatings, membranes) that encapsulates, solubilizes, or otherwise acts as a vehicle for packaging the therapeutic agent. In such conventional systems, the release profile of the therapeutic agent is controlled primarily or entirely by the characteristics (e.g., biodegradability, hydrophilicity, hydrophobicity, porosity) of the carrier material. In contrast, the implantable depots described herein can be provided without any carrier material. Instead, the majority of the implantable depot—or even the entirety of the implantable depot—can be made of the therapeutic agent. Accordingly, the release profile of the therapeutic agent can be controlled primarily or entirely by the characteristics of the therapeutic agent itself. In some embodiments, for example, the therapeutic agent is provided in a hydrophobic, free base form that, when implanted in vivo, provides sustained, controlled release over an extended time period. Thus, compared to conventional systems, the implantable depots described herein can have higher therapeutic payloads, smaller implant sizes, and/or reduced manufacturing costs and complexity.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

As used herein, the terms “vertical,” “lateral,” “upper,” and “lower” can refer to relative directions or positions of features of the embodiments disclosed herein in view of the orientation shown in the Figures. For example, “upper” or “uppermost” can refer to a feature positioned closer to the top of a page than another feature. These terms, however, should be construed broadly to include embodiments having other orientations, such as inverted or inclined orientations where top/bottom, over/under, above/below, up/down, and left/right can be interchanged depending on the orientation.

The headings provided herein are for convenience only and do not interpret the scope or meaning of the claimed present technology. Embodiments under any one heading may be used in conjunction with embodiments under any other heading.

### I. Implantable Depots

### A. Composition

The present technology provides implantable depots that are configured to be positioned at a treatment site in a patient's body to release at least one therapeutic agent at the treatment site in a controlled manner. The therapeutic agent can be any biologically active substance (or combination of substances) that provides a therapeutic effect in a patient in need thereof. As used herein, “therapeutic agent” or “drug” may refer to a single therapeutic agent, or may refer to a combination of therapeutic agents. In some embodiments, the therapeutic agent includes only a single therapeutic agent. In other embodiments, the therapeutic agent can include two or more therapeutic agents for simultaneous or sequential release.

For example, the therapeutic agent can be or include an analgesic for addressing postoperative pain and/or other types of pain (e.g., chronic pain). The terms “analgesic” and “analgesic agent” are used interchangeably herein to refer to one or more local or systemic agents that are administered to reduce, prevent, alleviate, or remove pain entirely, such as systemic and/or local anesthetics, narcotics, and/or anti-inflammatory agents. Additional details and examples of analgesics suitable for use in the present technology are provided below.

As described above, the implantable depot can include a relatively high loading of the therapeutic agent, compared to conventional delivery systems. For example, the therapeutic agent can constitute a majority of the total mass of the implantable depot, such as at least 50%, 55%, 60%, 65%, 70%, 75%, 80%, 85%, 90%, 95%, 99%, 99.5%, or 100% of the total mass of the implantable depot. Alternatively or in combination, the therapeutic agent can be no more than 99.5%, 99%, 95%, 90%, 85%, 80%, 75%, 70%, 65%, 60%, or 55% of the total mass of the implantable depot.

In some embodiments, the depots described herein have a total mass (e.g., total dry mass) within a range from 100 mg to 1500 mg, 100 mg to 1000 mg, 100 mg to 500 mg, 300 mg to 500 mg, 500 mg to 1000 mg, or 800 mg to 1000 mg. For example, the total mass can be greater than or equal to 100 mg, 150 mg, 200 mg, 250 mg, 300 mg, 350 mg, 400 mg, 425 mg, 450 mg, 475 mg, 500 mg, 525 mg, 550 mg, 575 mg, 600 mg, 650 mg, 700 mg, 750 mg, 800 mg, 825 mg, 850 mg, 875 mg, 900 mg, 925 mg, 950 mg, 975 mg, or 1000 mg.

The total mass of the therapeutic agent within the depot can be within a range from 100 mg to 1800 mg, 100 mg to 1500 mg, 100 mg to 1000 mg, 200 mg to 800 mg, 300 mg to 600 mg, 500 mg to 700 mg, 540 mg to 660 mg, or 570 mg to 630 mg. In some embodiments, the total mass of the therapeutic agent within an individual depot is greater than or equal to 25 mg, 50 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, or 1800 mg.

Suitable dosage ranges utilizing the implantable depots of the present technology are dependent on the potency of the particular therapeutic agent, but can be within a range from 0.001 mg to 500 mg of therapeutic agent per kg body weight, for example, within a range from 0.1 mg to 200 mg per kg body weight, or within a range from 1 mg to 100 mg per kg body weight. Dosage ranges can be readily determined by methods known to one skilled in the art. Dosage unit forms can contain between about 1 mg to about 500 mg of the therapeutic agent.

In some embodiments, the implantable depot does not include any carrier material (e.g., polymers, dendrimers, cyclodextrins, liposomes, lipids, coatings, membranes) configured to package the therapeutic agent into a controlled release vehicle. Carrier materials found in conventional drug delivery systems may package their therapeutic payloads in various ways, including, but not limited to: encapsulating the therapeutic agent into a particle, liposome, matrix, or scaffold; dissolving the therapeutic agent in a liquid or solid medium; forming an emulsion or suspension containing the therapeutic agent; and/or providing a barrier that partially or fully isolates the therapeutic agent from the surrounding environment. In such systems, the release profile of the therapeutic agent may therefore be determined primarily or entirely by the properties of the carrier material, such as the biodegradability, hydrophilicity, hydrophobicity, and/or porosity of the carrier material. In contrast, the implantable depots described herein can be provided without any carrier materials for the therapeutic agent, such that the release profile of the therapeutic agent is determined primarily or entirely by the properties of the therapeutic agent itself, as described further below. Alternatively, the implantable depot can include materials that would conventionally be considered to be carrier materials, but are not functioning as carrier materials in the depot (e.g., they are not being used to package the therapeutic agent for controlled release). In such embodiments, the amount of such “carrier materials” in the implantable depot may be much lower compared to conventional drug delivery systems, e.g., the “carrier material” can be no more than 20%, 15%, 10%, 9%, 8%, 7%, 6%, 5%, 4%, 3%, 2%, or 1% of the total mass of the implantable depot.

The properties of the therapeutic agent can be selected to provide a desired release profile in vivo. For example, the therapeutic agent can be sufficiently hydrophobic to elute from the depot in a controlled, sustained manner when exposed to physiologic fluids at a treatment site in vivo. Accordingly, the controlled, sustained release of the therapeutic agent can be achieved without using any carrier materials to slow the release rate of the therapeutic agent after implantation in vivo.

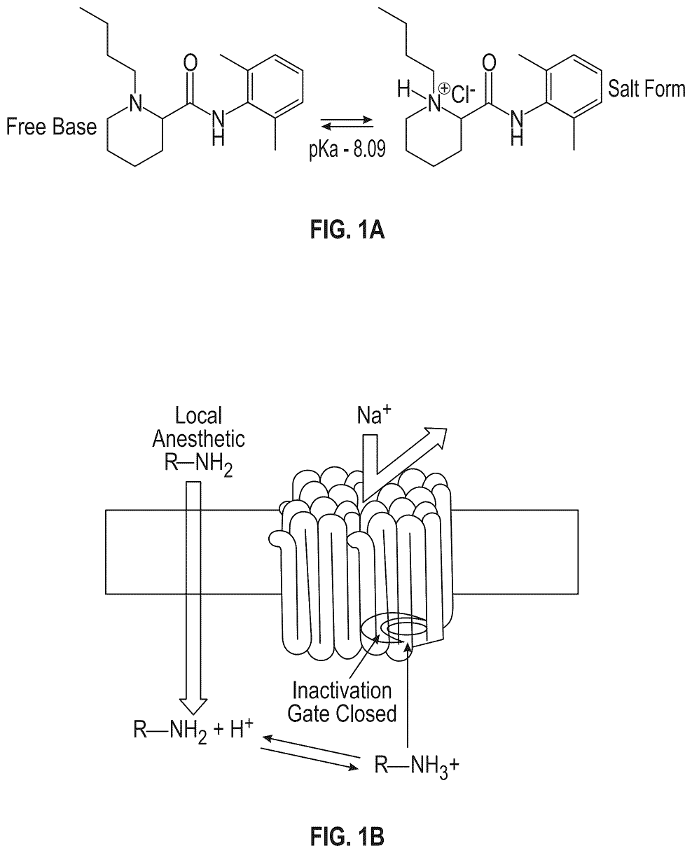

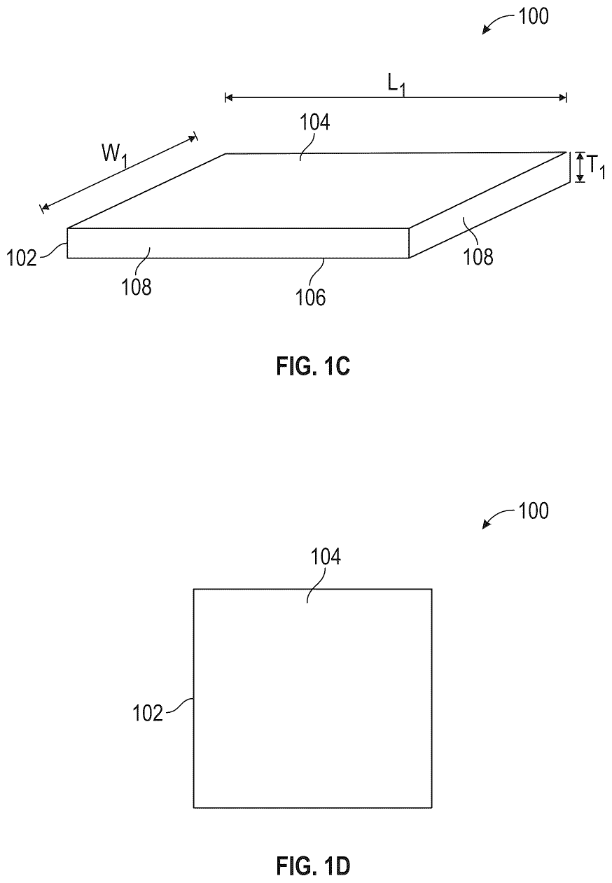

In some embodiments, the therapeutic agent has multiple forms with varying degrees of hydrophobicity, such as at least one hydrophobic form and at least one hydrophilic form. For example, the therapeutic agent can be or include an amine compound having a hydrophobic free base form and a hydrophilic salt form. The amine compound can be an amine-containing analgesic, such as an amino amide local anesthetic (e.g., bupivacaine, ropivacaine, lidocaine, mepivacaine, prilocaine, etidocaine, levobupivacaine, trimecaine, articaine) or an amino ester local anesthetic (e.g., benzocaine, procaine, tetracaine, chloroprocaine). The amine-containing analgesic can have a free base form (e.g., bupivacaine free base, ropivacaine free base) in which the amine group is deprotonated, and a salt form (e.g., bupivacaine hydrochloride, bupivacaine hydrochloride monohydrate, ropivacaine hydrochloride, ropivacaine hydrochloride monohydrate) in which the amine is protonated and associated with a counterion (e.g., chloride, bromide, sulfate, phosphate, nitrate, acetate, oxalate, citrate, tartrate). For example, FIG. 1A illustrates the free base form and hydrochloride salt form of bupivacaine (pKa=8.09). The amine-containing analgesic can contain salt forms of varying counterion combinations that alter the hydrophobicity and/or dissolution rate of the amine-containing analgesic.

In some embodiments, the hydrophobic (e.g., free base) form is the nonactive form of the therapeutic agent, while the hydrophilic (e.g., salt form) of the therapeutic agent is the active form of the therapeutic agent. For example, FIG. 1B illustrates cellular uptake of an amine-containing local anesthetic. The protonated, salt form of the amine-containing local anesthetic is the active form that, upon intracellular binding to voltage-gated sodium ion channels, blocks sodium ion influx and thus prevents depolarization and inhibits pain signaling. However, the deprotonated, free base form of the amine-containing local anesthetic may cross the cellular membrane via passive diffusion more easily than the salt form due to its increased hydrophobicity. In embodiments where the pKa of the amine-containing local anesthetic is higher than the intracellular pH, a proportion of the amine-containing local anesthetic can spontaneously convert from the free base form to the salt form upon entering the intracellular environment. Accordingly, implantable depots that are formulated mostly or entirely with the nonactive, free base form of the amine-containing local anesthetic can still provide high therapeutic efficacy.

The therapeutic agent in the implantable depot can be provided partially or entirely in the hydrophobic (e.g., free base) form. For example, at least 10%, 20%, 30%, 40%, 50%, 55%, 60%, 65%, 70%, 75%, 80%, 85%, 90%, 95%, 99%, 99.5%, or 100% of the therapeutic agent by mass can be in the hydrophobic form. Alternatively or in combination, no more than 95%, 90%, 85%, 80%, 75%, 70%, 65%, 60%, 55%, 50%, 40%, 30%, or 20% of the therapeutic agent by mass can be in the hydrophobic form.

Optionally, the implantable depot can include a combination of a hydrophobic (e.g., free base) form and a hydrophilic (e.g., salt) form of the therapeutic agent. The relative amounts of the hydrophobic form and the hydrophilic form can be selected to produce a desired release profile, e.g., increasing the relative amount of the hydrophobic form can produce a slower release rate, while increasing the relative amount of the hydrophilic form can produce a faster release rate. Increasing the relative amount of the hydrophilic form can also be used to achieve a bolus release of the therapeutic agent immediately after implantation, for example, to address the higher degree of pain experienced by patients in the hours and/or days immediately following surgery (as compared to several days or a week after surgery). In some embodiments, the ratio of the total mass of the hydrophobic form to the total mass of the hydrophilic form is greater than or equal to 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, or 20:1. In some embodiments, at least 10%, 20%, 30%, 40%, 50%, 55%, 60%, 65%, 70%, 75%, 80%, 85%, or 90% of the therapeutic agent by mass is in the hydrophilic form; and/or no more than 95%, 90%, 85%, 80%, 75%, 70%, 65%, 60%, 55%, 50%, 40%, 30%, 20%, or 10% of the therapeutic agent by mass is in the hydrophilic form.

Alternatively or in combination, the implantable depots described herein can include other types of therapeutic agents. In some embodiments, the therapeutic agent includes narcotics, for example, cocaine or anti-inflammatory agents. Examples of appropriate anti-inflammatory agents include steroids, such as prednisone, betamethasone, cortisone, dexamethasone, hydrocortisone, and methylprednisolone. Other appropriate anti-inflammatory agents include non-steroidal anti-inflammatory drugs (NSAIDs), such as aspirin, ibuprofen, naproxen sodium, diclofenac, diclofenac-misoprostol, celecoxib, piroxicam, indomethacin, meloxicam, ketoprofen, sulindac, diflunisal, nabumetone, oxaprozin, tolmetin, salsalate, etodolac, fenoprofen, flurbiprofen, ketorolac, meclofenamate, mefenamic acid, and other COX-2 inhibitors, and combinations thereof.

In some embodiments, the therapeutic agent is or includes an antibiotic, an antimicrobial or antifungal agent, or combinations thereof. For example, suitable antibiotics and antimicrobials include, but are not limited to, amoxicillin, amoxicillin/clavulanate, cephalexin, ciprofloxacin, clindamycin, metronidazole, azithromycin, levofloxacin, sulfamethoxazole/trimethoprim, tetracycline, minocycline, tigecycline, doxycycline, rifampin, triclosan, chlorhexidine, penicillin, aminoglycides, quinolones, fluoroquinolones, vancomycin, gentamycin, cephalosporin, carbapenem, imipenem, ertapenem, antimicrobial peptides, cecropin-mellitin, magainin, dermaseptin, cathelicidin, α-defensins, and α-protegrins. Antifungal agents include, but are not limited to, ketoconazole, clortrimazole, miconazole, econazole, intraconazole, fluconazole, bifoconazole, terconazole, butaconazole, tioconazole, oxiconazole, sulconazole, saperconazole, voriconazole, terbinafine, amorolfine, naftifine, griscofulvin, haloprogin, butenafine, tolnaftate, nystatin, cyclohexamide, ciclopirox, flucytosine, terbinafine, and amphotericin B.

In some embodiments, the therapeutic agent is or includes an adrenocorticostatic, a β-adrenolytic, an androgen or antiandrogen, an antianemic, an antiparasitic, an anabolic, an anesthetic or analgesic, an analeptic, an antiallergic, an antiarrhythmic, an anti-arteriosclerotic, an antibiotic, an antidiabetic, an antifibrinolytic, an anticonvulsive, an angiogenesis inhibitor, an anticholinergic, an enzyme, a coenzyme or a corresponding inhibitor, an antihistaminic, an antihypertensive, an antihypotensive, an anticoagulant, an antimycotic, an antiseptic, an anti-infective, an antihemorrhagic, a β-receptor antagonist, a calcium channel antagonist, an antimyasthenic, an antiphlogistic, an antipyretic, an antirheumatic, a cardiotonic, a chemotherapeutic, a coronary dilator, a cytostatic, a glucocorticoid, a hemostatic, an immunoglobulin or its fragment, a chemokine, a cytokine, a mitogen, a cell differentiation factor, a cytotoxic agent, a hormone, an immunosuppressant, an immunostimulant, a morphine antagonist, an muscle relaxant, a narcotic, a vector, a peptide, a (para)sympathicomimetic, a (para)sympatholytic, a protein, a cell, a selective estrogen receptor modulator (SERM), a sedating agent, an antispasmodic, a substance that inhibits the resorption of bone, a vasoconstrictor or vasodilator, a virustatic, or a wound-healing agent. In some embodiments, the therapeutic agent can include a hemostatic agent (e.g., aluminum sulfate, fibrin, micronized gelfoam, etc.), which can be especially beneficial when implanting the depot in areas with high vascular flow and potentially above-average post-operative bleeding (e.g., thoracic region, abdominal region, anorectal region, head and neck region, etc.).

In some embodiments, the therapeutic agent is or includes a drug used in the treatment of cancer or a pharmaceutically acceptable salt thereof. Such chemotherapeutic agents include antibodies, alkylating agents, angiogenesis inhibitors, antimetabolites, DNA cleavers, DNA crosslinkers, DNA intercalators, DNA minor groove binders, enediynes, heat shock protein 90 inhibitors, histone deacetylase inhibitors, immunomodulators, microtubule stabilizers, nucleoside (purine or pyrimidine) analogs, nuclear export inhibitors, proteasome inhibitors, topoisomerase (I or II) inhibitors, tyrosine kinase inhibitors, and serine/threonine kinase inhibitors. Specific therapeutic agents include, but are not limited to, adalimumab, ansamitocin P3, auristatin, bendamustine, bevacizumab, bicalutamide, bleomycin, bortezomib, busulfan, callistatin A, camptothecin, capecitabine, carboplatin, carmustine, cetuximab, cisplatin, cladribin, cytarabin, cryptophycins, dacarbazine, dasatinib, daunorubicin, docetaxel, doxorubicin, duocarmycin, dynemycin A, epothilones, etoposide, floxuridine, fludarabine, 5-fluorouracil, gefitinib, gemcitabine, ipilimumab, hydroxyurea, imatinib, infliximab, interferons, interleukins, beta-lapachone, lenalidomide, irinotecan, maytansine, mechlorethamine, melphalan, 6-mercaptopurine, methotrexate, mitomycin C, nilotinib, oxaliplatin, paclitaxel, procarbazine, suberoylanilide hydroxamic acid (SAHA), 6-thioguanidine, thiotepa, teniposide, topotecan, trastuzumab, trichostatin A, vinblastine, vincristine, vindesine, and tamoxifen.

In some embodiments, the therapeutic agent is or includes a botulinum toxin or other neurotoxin used in the treatment of various neuromuscular and/or neuroglandular disorders and neuropathies associated with pain. The botulinum toxin or other neurotoxin can include the pharmacologically active drug or a pharmaceutically acceptable salt thereof. The botulinum toxin can be selected from a variety of strains of Clostridium botulinum and may comprise the pharmacologically active drug or a pharmaceutically acceptable salt thereof. In some embodiment, the botulinum toxin is selected from the group consisting of botulinum toxin types A, B, C, D, E, F, and G.

A pharmaceutically acceptable salt refers to those salts that retain the biological effectiveness and properties of neutral therapeutic agents and that are not otherwise unacceptable for pharmaceutical use. Pharmaceutically acceptable salts include salts of acidic or basic groups, which groups may be present in the therapeutic agents. The therapeutic agents used in the present technology that are basic in nature are capable of forming a wide variety of salts with various inorganic and organic acids. Pharmaceutically acceptable acid addition salts of basic therapeutic agents used in the present technology can include those that form non-toxic acid addition salts, e.g., salts comprising pharmacologically acceptable anions, such as hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (1,1′-methylene-bis-(2-hydroxy-3-naphthoate)) salts. The therapeutic agents of the present technology that include an amino moiety can form pharmaceutically acceptable salts with various amino acids, in addition to the acids mentioned above. Suitable base salts can be formed from bases which form non-toxic salts, and can include aluminum, calcium, lithium, magnesium, potassium, sodium, zinc, or diethanolamine salts. A pharmaceutically acceptable salt can include another molecule, such as water or another biologically compatible solvent (a solvate), an acetate ion, a succinate ion, or other counterion. The counterion can be any organic or inorganic moiety that stabilizes the charge on the parent compound. Optionally, a pharmaceutically acceptable salt can include multiple counterions.

The therapeutic agent can be micronized, jet milled, and/or passed through a sieve to form consistent particle sizes, which can further facilitate the controlled release of the therapeutic agent. This process can be helpful for highly insoluble therapeutic agents, for example. Moreover, the particle size can affect the release rate of the therapeutic agent, e.g., smaller particle sizes can correlate to faster release rates. In some embodiments, the particle size of the therapeutic agent (e.g., the DS50 value) is less than or equal to 500 μm, 450 μm, 400 μm, 350 μm, 300 μm, 250 μm, 200 μm, 150 μm, 100 μm, 90 μm, 80 μm, 70 μm, 60 μm, 50 μm, 40 μm, 30 μm, 20 μm, 15 μm, 14 μm, 13 μm, 12 μm, 11 μm, 10 μm, 9 μm, 8 μm, 7 μm, 6 μm, 5 μm, 4 μm, 3 μm, 2 μm, or 1 μm. In some embodiments, the particle size is within a range from 1 μm to 500 μm, 1 μm to 200 μm, 1 μm to 100 μm, 1 μm to 50 μm, 1 μm to 10 μm, 10 μm to 500 μm, 10 μm to 200 μm, 10 μm to 100 μm, 10 μm to 50 μm, 50 μm to 500 μm, 50 μm to 200 μm, 50 μm to 100 μm, 100 μm to 500 μm, 100 μm to 200 μm, or 200 μm to 500 μm.

In some embodiments, the implantable depot is configured to release multiple therapeutic agents in a simultaneous or sequential manner, e.g., to provide added clinical benefits. For example, in the context of pain management, the depot can release a first analgesic having a faster onset (e.g., lidocaine) and a second analgesic having a slower onset (e.g., bupivacaine). As another example, the depot can release a first therapeutic agent having a first type of therapeutic effect (e.g., an analgesic effect), and a second therapeutic agent having a second type of therapeutic effect (e.g., increasing or decreasing blood flow, reducing inflammation, altering water uptake, affecting pH within the depot and/or in the surrounding environment, having antibacterial properties, having bone healing properties, etc.). The second therapeutic agent can enhance the efficacy of the first therapeutic agent or can independently provide a therapeutic benefit for the patient. The implantable depots described herein can include any suitable number of therapeutic agents, such as one, two, three, four, five, or more different therapeutic agents. The first and second therapeutic agents, and any other therapeutic agents, can be any of the therapeutic agents disclosed herein.

In some embodiments, the implantable depots described herein are not made entirely out of the therapeutic agent, but instead include one or more additional components. For example, the implantable depots herein can include at least one excipient configured to modulate the release profile of the therapeutic agent. The implantable depots can include any suitable number of excipients, such as one, two, three, four, five, or more different excipients. In some embodiments, the excipient is not considered to be a carrier material, in that the excipient does not package the therapeutic agent into a controlled release vehicle, as discussed above. Instead, the excipient can increase or decrease the release rate of the therapeutic agent by altering the structure and/or properties of the implantable depot.

For example, the excipient can increase the release rate of the therapeutic agent by increasing the porosity of the implantable depot. In such embodiments, the excipient can be a hydrophilic substance (e.g., a water-soluble small molecule) that, when exposed to physiologic fluids at the treatment site, elutes out of the implantable depot to form voids within the bulk material of the depot (e.g., within the therapeutic agent). The voids can promote diffusion of physiologic fluids into the depot and/or diffusion of the therapeutic agent out of the depot, thus causing faster elution of the therapeutic agent. Alternatively or in combination, the voids can decrease the aggregation and/or crystallinity of the therapeutic agent, which can also enhance release. Conversely, a hydrophobic excipient can be used to reduce the release rate of the therapeutic agent (e.g., a therapeutic agent in a hydrophobic free base form). Optionally, the excipient can provide other benefits, such as enhancing adhesion of the implantable depot to biological tissues, reducing and/or constricting blood flow (e.g., epinephrine to reduce blood flow via vasoconstriction), modifying the pH of the site of tissue injury to render the therapeutic agent more effective (e.g., meloxicam or other agents to raise the pH at the implantation site to a pKa of the therapeutic agent), improving fluoroscopic visualization of the depot (e.g., via improving a radiopacity of the depot), fighting infection (e.g., such as silver nanoparticles), enhancing tissue repair and healing, etc.

The excipient can be a polymeric material, a non-polymeric material, or a combination thereof. Examples of excipients suitable for use in the present technology include, but are not limited to: polysorbates (e.g., Polysorbate 80, Polysorbate 60, Polysorbate 40, or Polysorbate 20 (Tween 20™)), polyethylene glycol, polyvinylpyrrolidone, poly (lactide-co-glycolide), salts (e.g, NaCl), saccharides (e.g., sucrose), sorbitan fatty acid esters (e.g., sorbitan monostearate (Span 60), sorbitan tristearate (Span 65), sorbitan trioleate (Span 85), sorbitan monooleate (Span 80), sorbitan monopalmitate, sorbitan monolaurate, sorbitan monopalmitate, sorbitan tribehenate), sucrose esters (e.g., sucrose monodecanoate, sucrose monolaurate, sucrose distearate, sucrose stearate), castor oils (e.g., polyethoxylated castor oil, polyoxyl hydrogenated castor oil, Polyoxyl 35 castor oil, Polyoxyl 40 Hydrogenated castor oil, Polyoxyl 40 castor oil, Cremophor® RH60, Cremophor® RH40), polyethylene glycol ester glycerides (e.g., Labrasol®, Labrifil® 1944), poloxamers, polyoxyethylene polyoxypropylene 1800, polyoxyethylene fatty acid esters (e.g., Polyoxyl 20 Stearyl Ether, diethylene glycol octadecyl ether, glyceryl monostearate, triglycerol monostearate, Polyoxyl 20 stearate, Polyoxyl 40 stearate, polyoxyethylene sorbitan monoisostearate, polyethylene glycol 40 sorbitan diisostearate), oleic acid, sodium desoxycholate, sodium lauryl sulfate, myristic acid, stearic acid, vitamin E D-alpha-tocopherol polyethylene glycol succinate (vitamin E-TPGS), saturated polyglycolized glycerides (e.g., Gelucire® 44/14, Gelucirc® 50/13), polypropoxylated stearyl alcohols (e.g., Acconon® MC-8, Acconon® CC-6), sugar alcohols (e.g., sorbitol), cellulose compounds (e.g., methyl cellulose, hydroxypropyl methylcellulose), or derivatives or combinations thereof.

In some embodiments, the excipient is or includes a plasticizer that increases the flexibility of the depot, makes it easier to form the depot into a desired shape, and/or reduces friction on one or more surfaces of the depot. The plasticizer can be a non-volatile or low-volatility liquid, or a solid substance. The plasticizer can have any suitable molecular weight, such as a molecular weight less than or equal to 20 kDa, 10 kDa, 5 kDa, 2 kDa, 1 kDa, 900 Da, 800 Da, 700 Da, 600 Da, 500 Da, 400 Da, 300 Da, 200 Da, or 100 Da. The plasticizer can be or include a hydrophobic substance, such as a triglyceride (e.g., Miglyol, tricaprilin), a fatty acid ester (e.g., ethyl hexanoate, ethyl oleate, isopropyl palmitate, isopropyl myristate), a lactic acid ester (e.g., lactic acid dodecyl ester), a citrate (e.g., acetyltricthyl citrate, tributyl citrate, acetyltributyl citrate), diethyl phthalate (DEP), dibutyl sebacate, glycerol triacetate, acetylated monoglyceride, or benzyl benzoate. The plasticizer can be or include a water-soluble substance, such as triethyl citrate, a polyethylene glycol, a polysorbate, a propylene glycol, triacetin, benzyl alcohol, glycerol formal, or ethyl lactate.

In some embodiments, the excipient constitutes no more than 50%, 20%, 15%, 10%, 9%, 8%, 7%, 6%, 5%, 4.5%, 4%, 3.5%, 3%, 2.5%, 2%, 1.5%, 1%, 0.75%, 0.5%, 0.25%, or 0.1% of the total mass of the implantable depot. For example, the total mass of the excipient can be within a range from 0.1% to 20%, 0.5% to 10%, or 1% to 5% of the total mass of the depot. The total mass of the excipient in the depot can be within a range from 1 mg to 200 mg, 10 mg to 100 mg, 10 mg to 50 mg, 20 mg to 50 mg, 20 mg to 40 mg, or 25 mg to 35 mg. In some embodiments, the total mass of the excipient is less than or equal to 200 mg, 150 mg, 100 mg, 90 mg, 80 mg, 70 mg, 60 mg, 50 mg, 45 mg, 40 mg, 35 mg, 30 mg, 25 mg, 20 mg, 15 mg, 10 mg, 5 mg, or 1 mg.

### B. Geometry and Methods of Manufacturing

The implantable depots of the present technology can be provided in any form factor suitable for being implanted at a treatment site and controllably releasing the therapeutic agent. For example, the implantable depot can be configured as a film, sheet, strip, ribbon, capsule, coating, matrix, wafer, pill, pellet, bead, scaffold, powder, fiber, filament, particle, or a combination thereof. The geometry of the implantable depot can be selected based on various parameters, such as the desired release profile of the therapeutic agent and/or the location at which the depot is to be implanted.

FIGS. 1C and 1D are perspective and top views, respectively, of an implantable depot 100 configured in accordance with embodiments of the present technology. The depot 100 includes a solid, monolithic body 102 having a square or rectangular shape. The body 102 can be made out of at least one therapeutic agent (e.g., a therapeutic agent in a hydrophobic, free base form and/or in a hydrophilic, salt form) and, optionally, one or more excipients, as described above in Section I.A. In some embodiments, the body 102 has a plurality of surfaces (e.g., an upper surface 104, a lower surface 106, and/or lateral surfaces 108), some or all of which are directly exposed to physiologic fluids when the depot 100 is implanted at a treatment in vivo. The interactions between the physiologic fluids, therapeutic agent, and excipient (if present) can cause the therapeutic agent to elute out of the depot 100 and into the surrounding environment according to a desired release profile.

The dimensions of the depot 100 can be varied as desired. For example, the depot 100 can have a length L₁ within a range from 1 mm to 100 mm, 5 mm to 75 mm, 10 mm to 50 mm, 15 mm to 45 mm, 20 mm to 30 mm, 25 mm to 35 mm, 1 mm to 20 mm, or 15 mm to 25 mm. In some embodiments, the length L₁ is at least 1 mm, 5 mm, 10 mm, 15 mm, 20 mm, 25 mm, 30 mm, 35 mm, 40 mm, 45 mm, or 50 mm. The depot 100 can have a width W₁ from 1 mm to 100 mm, 5 mm to 75 mm, 10 mm to 50 mm, 15 mm to 45 mm, 20 mm to 30 mm, 25 mm to 35 mm, 1 mm to 20 mm, or 15 mm to 25 mm. In some embodiments, the width W₁ is at least 1 mm, 5 mm, 10 mm, 15 mm, 20 mm, 25 mm, 30 mm, 35 mm, 40 mm, 45 mm, or 50 mm. The depot 100 can have a thickness T₁ within a range from a range from 100 μm to 5 mm, 500 μm to 2.5 mm, 1 mm to 2 mm, 750 μm to 1.25 mm, 1 mm to 1.5 mm, 1.25 mm to 1.75 mm, 1.75 mm to 2.25 mm, or 2 mm to 2.5 mm. For example, the thickness T₁ can be greater than or equal to 100 μm, 200 μm, 300 μm, 400 μm, 500 μm, 600 μm, 700 μm, 800 μm, 900 μm, 1 mm, 1.1 mm, 1.2 mm, 1.25 mm, 1.3 mm, 1.4 mm, 1.5 mm, 1.6 mm, 1.7 mm, 1.75 mm, 1.8 mm, 1.9 mm, 2 mm, 2.1 mm, 2.2 mm, 2.25 mm, 2.3 mm, 2.4 mm, 2.5 mm, 2.6 mm, 2.7 mm, 2.75 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, 5 mm, 6 mm, 7 mm, 8 mm, 9 mm, 10 mm, 11 mm, 12 mm, 13 mm, 14 mm, or 15 mm. Alternatively or in combination, the thickness T₁ can be less than or equal to 15 mm, 14 mm, 13 mm, 12 mm, 11 mm, 10 mm, 9 mm, 8 mm, 7 mm, 6 mm, 5 mm, 4.9 mm, 4.8 mm, 4.7 mm, 4.6 mm, 4.5 mm, 4.4 mm, 4.3 mm, 4.2 mm, 4.1 mm, 4 mm, 3.9 mm, 3.8 mm, 3.7 mm, 3.6 mm, 3.5 mm, 3.4 mm, 3.3 mm, 3.2 mm, 3.1 mm, 3 mm, 2.9 mm, 2.8 mm, 2.75 mm, 2.7 mm, 2.6 mm, 2.5 mm, 2.4 mm, 2.3 mm, 2.25 mm, 2.2 mm, 2.1 mm, 2 mm, 1.9 mm, 1.8 mm, 1.75 mm, 1.7 mm, 1.6 mm, 1.5 mm, 1.4 mm, 1.3 mm, 1.25 mm, 1.2 mm, 1.1 mm, 1 mm, 900 μm, 800 μm, 700 μm, 600 μm, 500 μm, 400 μm, 300 μm, 200 μm, or 100 μm.

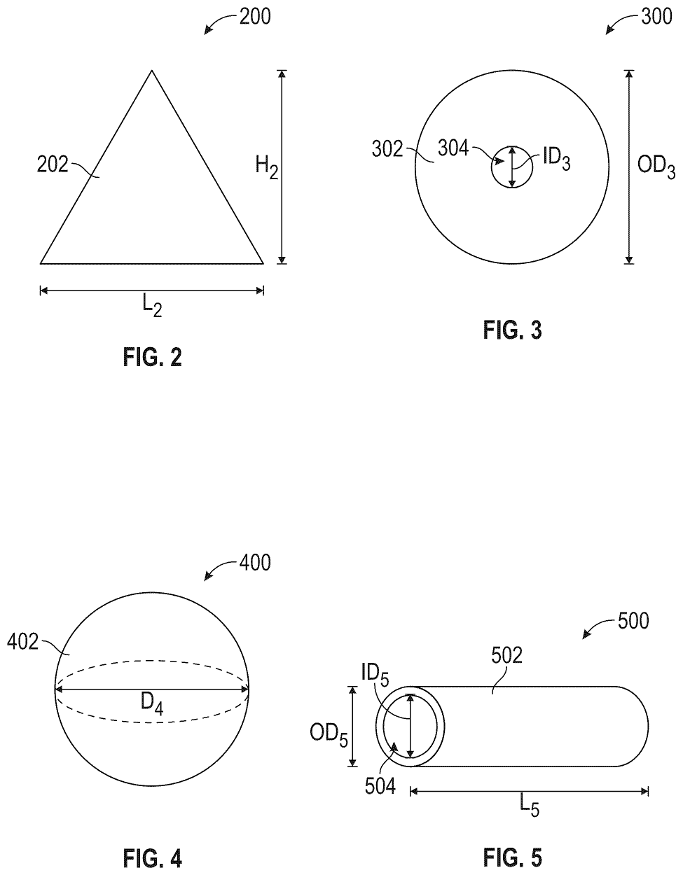

FIG. 2 is a top view of a triangular implantable depot 200 configured in accordance with embodiments of the present technology. The depot 200 can be generally similar to the depot 100 of FIGS. 1C and 1D, except that the body 202 of the depot 200 has a triangular shape. The triangular shape may be advantageous for conforming to the shape of certain surgical sites, such as the femoral gutters and/or suprapatellar pouch of the knee. In the illustrated embodiment, the depot 200 is shaped as an equilateral triangle, such that all three sides of the depot 200 have the same length L₂. The length L₂ can be within a range from 1 mm to 100 mm, 10 mm to 50 mm, 15 mm to 45 mm, 20 mm to 30 mm, or 25 mm to 35 mm. In some embodiments, the length L₂ is at least 10 mm, 15 mm, 20 mm, 25 mm, 30 mm, 35 mm, 40 mm, 45 mm, or 50 mm. In other embodiments, however, some or all of the sides of the depot 200 can have different respective lengths. The depot 200 can have a height H₂ within a range from 10 mm to 40 mm, 15 mm to 35 mm, 20 mm to 30 mm, or 25 mm to 35 mm. In some embodiments, the height H₂ is greater than or equal to 10 mm, 15 mm, 20 mm, 25 mm, 30 mm, 35 mm, or 40 mm. The thickness of the depot 200 (not shown) can be the same or generally similar to the thickness T₁ of the depot 100 of FIGS. 1C and 1D.

FIG. 3 is a top view of a circular implantable depot 300 configured in accordance with embodiments of the present technology. The depot 300 can be generally similar to the depot 100 of FIGS. 1C and 1D, except that the body 302 of the depot 300 has a circular shape. The body 302 can have a diameter OD₃ within a range from 1 mm to 100 mm, 5 mm to 50 mm, 10 mm to 30 mm, or 10 mm to 15 mm. In some embodiments, the diameter OD₃ is at least 5 mm, 10 mm, 15 mm, 20 mm, 25 mm, 30 mm, 35 mm, 40 mm, 45 mm, or 50 mm. Optionally, the body 302 can include a central hole 304. The hole 304 can increase the release rate of the therapeutic agent by altering the surface area of the body 302 that is exposed to physiologic fluids and/or reducing the distance that the therapeutic agent travels to reach an exposed surface. In some embodiments, the hole 304 has a diameter ID₃ within a range from 1 mm to 20 mm, 2 mm to 15 mm, 5 mm to 10 mm, or 1 mm to 5 mm. For example, the diameter ID₃ can be less than or equal to 20 mm, 15 mm, 10 mm, 5 mm, 2 mm, or 1 mm. The thickness of the depot 300 (not shown) can be the same or generally similar to the thickness T₁ of the depot 100 of FIGS. 1C and 1D.

FIG. 4 is a perspective view of a spherical implantable depot 400 configured in accordance with embodiments of the present technology. The depot 400 has a body 402 having a spherical shape. The spherical shape may be advantageous for increasing the mechanical strength of the depot 400, as well as for packing irregularly-shaped spaces within a patient's body. For instance, a plurality of spherical depots 400 can be packed into an injury site (e.g., fracture site) or surgical site (e.g., joint replacement site). In some embodiments, the release rate of the therapeutic agent can be controlled by varying the size of the depot 400, e.g., a larger depot 400 may release the therapeutic agent at a slower rate than a smaller depot 400. For example, the diameter D₄ of the body 402 of the spherical depot 400 can be within a range from 1 mm to 100 mm, 5 mm to 75 mm, 10 mm to 50 mm, 15 mm to 45 mm, 20 mm to 30 mm, 25 mm to 35 mm, 1 mm to 10 mm, or 1 mm to 5 mm. In some embodiments, the diameter D₄ is at least 1 mm, 2 mm, 5 mm, 10 mm, 15 mm, 20 mm, 25 mm, 30 mm, 35 mm, 40 mm, 45 mm, or 50 mm.

FIG. 5 is a perspective of a cylindrical or tubular implantable depot 500 configured in accordance with embodiments of the present technology. The body 502 has a cylindrical shape with a length L₅ within a range from 1 mm to 100 mm, 10 mm to 50 mm, 15 mm to 45 mm, 20 mm to 30 mm, or 25 mm to 35 mm. In some embodiments, the length L₅ is at least 10 mm, 15 mm, 20 mm, 25 mm, 30 mm, 35 mm, 40 mm, 45 mm, or 50 mm. The diameter OD₅ of the body 502 can be within a range from 0.5 mm to 100 mm, 1 mm to 100 mm, 5 mm to 75 mm, 10 mm to 50 mm, 15 mm to 45 mm, 20 mm to 30 mm, 25 mm to 35 mm, 1 mm to 10 mm, 1 mm to 5 mm, 1 mm to 2 mm, 0.5 mm to 10 mm, 0.5 mm to 5 mm, 0.5 mm to 2 mm, or 0.5 mm to 1 mm. In some embodiments, the diameter OD₅ is at least 0.5 mm, 1 mm, 2 mm, 5 mm, 10 mm, 15 mm, 20 mm, 25 mm, 30 mm, 35 mm, 40 mm, 45 mm, or 50 mm. Alternatively or in combination, the diameter OD₅ can be less than or equal to 50 mm, 45 mm, 40 mm, 35 mm, 30 mm, 25 mm, 20 mm, 15 mm, 10 mm, 5 mm, 2 mm, 1 mm, or 0.5 mm. In some embodiments, the tubular depot 500 is sized to fit within a needle (e.g., a 12-gauge to 14-gauge needle), such that the depot 500 can be delivered to a treatment site via injection. Alternatively, the tubular depot 500 can be sized for delivery via a minimally invasive surgical tool such as a laparoscopic port. Moreover, thinner tubular depots 500 (e.g., having an outer diameter less than or equal to 5 mm, 2 mm, 1 mm, or 0.5 mm) may be beneficial for precisely targeting the therapeutic agent to certain anatomic structures such as the fascial plane, ligaments, etc. Multiple thin tubular depots 500 implanted at or proximate to a targeted anatomic structure can also be used to provide broader distribution of the therapeutic agent (e.g. extracapsular deposition for genicular nerves). In some embodiments, thinner tubular depots 500 are less painful to implant and thus can be delivered via injection without the need for an initial local anesthetic injection to numb the implantation site beforehand.

Optionally, the depot 500 can include a lumen 504 extending through the body 502, such that the body 502 is a hollow tube. The lumen 504 can increase the release rate of the therapeutic agent by altering the surface area of the body 502 that is exposed to physiologic fluids and/or reducing the distance that the therapeutic agent travels to reach an exposed surface. The diameter ID₅ of the lumen 504 can be within a range from 0.1 mm to 1 mm, 0.5 mm to 2 mm, 1 mm to 20 mm, 2 mm to 15 mm, 5 mm to 10 mm, or 1 mm to 5 mm. For example, the diameter ID₅ can be less than or equal to 20 mm, 15 mm, 10 mm, 5 mm, 2 mm, or 1 mm. In some embodiments, the geometry of the depot 500 is configured such that the depot 500 can be positioned around another tubular device, such as a catheter. In other embodiments, however, the lumen 504 can be omitted such that the body 502 is a solid tube.

The implantable depots herein can be provided in many different formats. In some embodiments, for example, the implantable depots herein are solid monolithic structures having a fixed shape. Accordingly, the depot can be placed in a treatment site in the patient's body without any changes to the shape of the depot. As described herein, the shape of the depot can be customized to the treatment site and/or delivery route, e.g., a triangular depot may be advantageous for placement in the femoral gutters and/or suprapatellar pouch of the knee, a spherical depot may be advantageous for packing a confined space, a tubular or cylindrical depot may be advantageous for delivery via a needle or a laparoscopic port, etc.

In some embodiments, the implantable depots described herein are moldable structures that are provided in an initial shape but can be formed into a different shape. For instance, the depots herein can be moldable at or near room temperature (e.g., 25° C.) and/or at or near physiological temperature (e.g., 37° C.). A moldable depot can be formed into a desired shape by hand and/or with the use of a tool (e.g., a mold, stamp, press). A moldable depot can be reshaped to conform to the patient's anatomy, which can be advantageous for fitting the depot into a confined space such as a bunionectomy site, a dental site, etc. In some embodiments, the moldable depot is provided in an initial shape having a predetermined dosage of the therapeutic agent (e.g., a maximum dosage), and the surgeon can reshape the depot and/or split the depot into smaller pieces for distribution across different treatment sites in the patient's body.

In some embodiments, the implantable depots described herein are provided in the form of a powder, rather than as a single monolithic structure. The individual particles of the powder can have any suitable size, such as an average particle diameter within a range from 1 μm to 100 μm, 10 μm to 75 μm, or 20 μm to 50 μm. The powder can be formed by grinding the therapeutic agent until the desired size range is achieved, e.g., using a mortar and pestle, or other suitable device. Optionally, sieving can be used to filter out particles having a larger and/or smaller size than the desired size range. In some embodiments, the powder is provided in a package having a predetermined dosage of the therapeutic agent (e.g., a maximum dosage), and the surgeon can pour the powder into or onto one or more treatment sites in the patient's body.

In some embodiments, the implantable depots herein are provided in the form of one or more filaments. The filaments can have any suitable diameter, such as a diameter less than or equal to 1 mm, 500 μm, 200 μm, 100 μm, 50 μm, 20 μm, 10 μm, 5 μm, or 1 μm. The filament diameter can be within a range from 1 μm to 1 mm, 1 μm to 500 μm, 1 μm to 200 μm, 1 μm to 100 μm, 1 μm to 50 μm, 1 μm to 20 μm, 1 μm to 10 μm, 1 μm to 5 μm, 1 μm to 2 μm, 5 μm to 1 mm, 5 μm to 500 μm, 5 μm to 200 μm, 5 μm to 100 μm, 5 μm to 50 μm, 5 μm to 20 μm, 5 μm to 10 μm, 10 μm to 1 mm, 10 μm to 500 μm, 10 μm to 200 μm, 10 μm to 100 μm, 10 μm to 50 μm, 10 μm to 20 μm, 20 μm to 1 mm, 20 μm to 500 μm, 20 μm to 200 μm, 20 μm to 100 μm, 20 μm to 50 μm, 50 μm to 1 mm, 50 μm to 500 μm, 50 μm to 200 μm, 50 μm to 100 μm, 100 μm to 1 mm, 100 μm to 500 μm, 100 μm to 200 μm, 200 μm to 1 mm, 200 μm to 500 μm, or 500 μm to 1 mm. The filaments can optionally be formed into a larger structure, such as a mesh, lattice, woven or twisted strand, foam, etc.

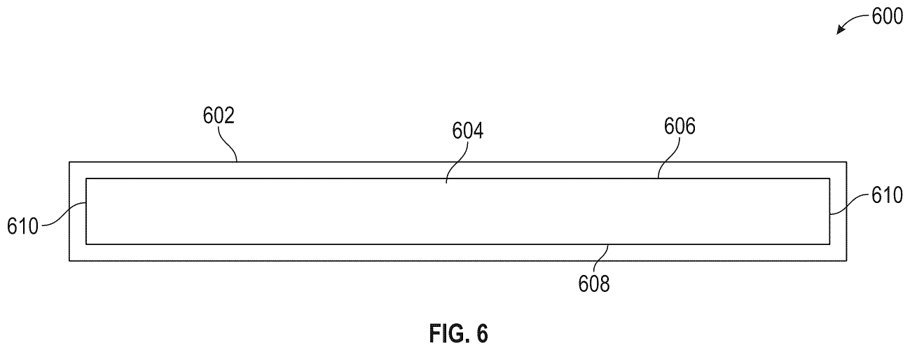

In some embodiments, the implantable depots herein are provided in the form of a coating. For example, FIG. 6 is a side cross-sectional view of an implantable depot 600 in the form of a coating 602 on a device 604, in accordance with embodiments of the present technology. The device 604 (shown schematically) can be any suitable medical device known to those of skill in the art, such as a mesh (e.g., hernia mesh), a prosthesis (e.g., knee prosthesis, shoulder prosthesis, hip prosthesis, breast prosthesis), an orthopedic implant (e.g., spinal implant, implants for long bone fractures), an antimicrobial implant (e.g., an antimicrobial pouch and/or mesh), a film (e.g., abdominal adhesion film), a catheter, a pacemaker, a stent (e.g., a cardiac stent, a vascular stent, a kidney stent, an esophageal stent, a urethral stent), a wound dressing (e.g., a bandage, gauze, a wrap), a wound closure device (e.g., a suture, a staple), an car device (e.g., an car plug, an car tube), a nasal splint, etc. The device 604 can be made out of metal, polymer (e.g., natural or synthetic polymers), ceramic, biological materials (e.g., tissue grafts, decellularized tissues), or a combination thereof.

The coating 602 can be made out of at least one therapeutic agent (e.g., a therapeutic agent in a hydrophobic, free base form and/or in a hydrophilic, salt form) and, optionally, one or more excipients, as described above in Section I.A. In the illustrated embodiment, the coating 602 is a single layer that covers all of the surfaces (e.g., upper surface 606, lower surface 608, and/or lateral surfaces 610) of the device 604. In other embodiments, however, the coating 602 can include multiple layers (e.g., two, three, four, five, or more layers), and/or can cover only some of the surfaces of the device 604 (e.g., the upper surface 606 only, the lower surface 608 only, the upper surface 606 and lower surface 608 only, the lateral surfaces 610 only, etc.). Additionally, although FIG. 6 illustrates the coating 602 as having a uniform thickness, in other embodiments, the coating 602 can have a variable thickness. The thickness of the coating 602 (e.g., average, maximum, or minimum thickness) can be within a range from 1 μm to 1 mm, 1 μm to 500 μm, 1 μm to 100 μm, 10 μm to 1 mm, 10 μm to 500 μm, 10 μm to 100 μm, 10 μm to 50 μm, or 500 μm to 1 mm. In some embodiments, the thickness can be no more than 1 mm, 900 μm, 800 μm, 700 μm, 600μ, 500 μm, 400 μm, 300 μm, 200 μm, 100 μm, 50 μm, 25 μm, 10 μm, 5 μm, or 1 μm. The coating 602 can be formed on the device 604 using any suitable technique, such as dip coating, spray coating, spin coating, deposition, or combinations thereof.

The implantable depots described herein can be manufactured in many different ways. In some embodiments, for example, an implantable depot (e.g., the depots 100-500 of FIGS. 1C-5) is manufactured using a melt form process in which the components of the depot (e.g., the therapeutic agent and any optional excipients) are provided in solid (e.g., particulate) form and are placed into a mold having a geometry which may or may not correspond to the desired final geometry of the depot. The excipient (if present) can be physically mixed in with the therapeutic agent. Alternatively, in embodiments where the therapeutic agent is provided in particulate format, the excipient can be used to coat the particles of the therapeutic agent, and the coated particles can then be placed into the mold. The depot components can be heated to a temperature sufficient to melt the components so they flow and fill the mold cavity. For example, the temperature can be at least 50° C., 60° C., 70° C., 80° C., 90° C., 100° C., 110° C., or 120° C. Subsequently the depot can be cooled within the mold, which may occur passively or actively (e.g., via refrigeration and/or other cooling devices). The depot can be cooled to room temperature (e.g., 25° C.) or any other suitable temperature (e.g., 4° C., 0° C.). After cooling, the depot can be removed from the mold. In some embodiments, the depot is still relatively malleable before fully cooled (e.g., when first removed from the mold), and thus can be remolded into various form factors (e.g., spheres, tubes, fibers, filaments, thin films).

As another example, an implantable depot can be manufactured using a 3D printing process, such as an extrusion-based printing process (e.g., fused deposition modeling (FDM)). The extrusion-based printing process can involve extruding the depot material (e.g., the therapeutic agent and any optional excipients) as a filament onto a build platform to form an individual layer of the depot. Optionally, the depot material can be heated and/or melted to facilitate extrusion of the depot material and/or to promote adhesion of the extruded material to previously deposited depot layers. Upon cooling, the depot material can solidify into the final geometry for the depot layer. This process can be repeated to build up the entire depot from a plurality of individual depot layers. 3D printing can be used to form depots with customized shapes and/or more complex geometries such as porous structures, infill patterns, etc.

In embodiments where the implantable depot is provided as a coating on a device (e.g., the depot 600 of FIG. 6), the coating can be formed by a dip coating process in which the device is immersed into a solution of the depot components (e.g., the therapeutic agent and any optional excipients). The depot solution can be formed, for example, by heating the depot components to a temperature of at least 50° C., 60° C., 70° C., 80° C., 90° C., 100° C., 110° C., or 120° C. The device can then be partially or completely immersed into the solution one or more times to form the coating on some or all of the surfaces of the device. The characteristics of the coating (e.g., thickness, roughness) can be controlled based on process parameters such as the number of dipping cycles, dipping speed, dipping angle, time spent immersed in the solution, solution temperature, solvent washes between dipping cycles, etc. Alternatively or in combination, the coating can be formed on the device using other types of processes such as spray coating, solvent casting, electrospinning, etc.

### C. Release Profile and Pharmacokinetics

The implantable depots of the present technology can be configured to deliver the therapeutic agent according to a desired release profile. As described elsewhere herein, the release profile of the depot can be tuned by adjusting the composition of the depot, such as the relative amounts of the hydrophobic and hydrophilic forms of the therapeutic agent, the amounts and/or types of excipients present, etc. The release profile can provide sustained, continuous release of the therapeutic agent over a desired release period or duration (e.g., the period after the depot is implanted in the body and/or immersed in fluid). The release period can be at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 40 days, 50 days, 60 days, 70 days, 90 days, 100 days, 200 days, 300 days, or 365 days. The depots herein can release at least 70%, 75%, 80%, 85%, 90%, 95%, 96%, 97%, 98%, 99%, or 100% of the initial amount (e.g., mass) of the therapeutic agent in the depot over the release period.

The release profile of a depot can be measured using in vitro or in vivo techniques. Any description herein of a release profile of a depot can refer to in vitro release, in vivo release, or both, unless otherwise specified. The release profile of a depot can be measured in vitro by immersing the depot in a suitable elution medium (e.g., phosphate-buffered saline) at a controlled temperature (e.g., 37° C.) and pH (e.g., 7.4, 5.8), and measuring the amount of released therapeutic agent at various time points (e.g., using spectrophotometric techniques). When measuring in vitro release, the elution pH and/or other parameters can be configured to approximate in vivo physiologic conditions (e.g., release is measured at pH 7.4). Alternatively, the elution pH and/or other parameters can be selected based on other considerations. For example, as a product advances in development or manufacturing, an accelerated in vitro release process can be developed, e.g., to facilitate quality control testing. The accelerated in vitro release can be accomplished through an increase in temperature, the addition of a surfactant or organic co-solvent to the aqueous buffer, and/or by a change in pH. For example, accelerated in vitro release can be measured at pH 5.8.

The release profile of a depot can be measured in vivo by implanting the depot at a treatment site in a subject (e.g., an animal or human subject), collecting local and/or systemic samples from the subject at various time points (e.g., blood samples, plasma samples, synovial fluid samples), and measuring the amount of therapeutic agent in the sample (e.g., using liquid chromatography tandem mass spectrometry). Optionally, a cumulative in vivo release profile can be estimated from concentration data by assuming that the total area under the curve (AUC_{0-inf}) of the concentration data corresponds to 100% release of the total therapeutic agent dose in the depot, then calculating the cumulative percentage release of the therapeutic agent at each study time point t₁ from the ratio of AUC₀₋ₜ₁ to AUC_{0-inf} normalized to 100%. As yet another example, the in vivo release profile can be determined by explanting the depot from the treatment site at various time points, and measuring the amount of therapeutic agent remaining in the depot. For example, the depot can be immersed in an extraction medium (e.g., 5:3 v/v acetonitrile: methanol) to dissolve the depot and release any remaining therapeutic agent. The extraction medium can be fully evaporated, and the therapeutic agent can be reconstituted using a suitable solvent (e.g., methanol). The reconstituted sample can be analyzed via high-performance liquid chromatography (HPLC) to measure the amount of therapeutic agent in the sample.

In some embodiment, the depots herein are configured to release the therapeutic agent at different rates over the treatment period. For example, the depots herein can release the therapeutic agent at a first rate during a first time period of the treatment, and a second rate during a second, subsequent time period of the treatment. For example, the first period can be the first 1 day, 2 days, 3 days, 4 days, 5 days, 6 days, 7 days, 8 days, 9 days, 10 days, 11 days, 12 days, 13 days, or 14 days of the treatment period; and the second period can be the next 1 day, 2 days, 3 days, 4 days, 5 days, 6 days, 7 days, 8 days, 9 days, 10 days, 11 days, 12 days, 13 days, or 14 days after the first period. Stated differently, the first period can be the first 1 hour, 2 hours, 5 hours, 10 hours, 12 hours, 20 hours, 24 hours, 30 hours, 36 hours, 40 hours, 48 hours, 50 hours, 60 hours, 70 hours, 72 hours, 80 hours, 84 hours, 90 hours, 96 hours, 100 hours, 108 hours, 120 hours, 150 hours, 200 hours, 250 hours, 300 hours, 350 hours, 400 hours, 450 hours, or 500 hours of the treatment period; and the second period can be the next 1 hour, 2 hours, 5 hours, 10 hours, 12 hours, 20 hours, 24 hours, 30 hours, 36 hours, 40 hours, 48 hours, 50 hours, 60 hours, 70 hours, 72 hours, 80 hours, 84 hours, 90 hours, 96 hours, 100 hours, 108 hours, 120 hours, 150 hours, 200 hours, 250 hours, 300 hours, 350 hours, 400 hours, 450 hours, or 500 hours of the treatment period after the first treatment period. The first rate may be the same as or different than (e.g., less than or greater than) the second rate. In some embodiments, the first rate is at least 2-fold, 3-fold, 4-old, 5-fold, 6-fold, 7-fold, 8-fold, 9-fold, or 10-fold greater than the second rate, or vice-versa.

In some embodiments, the depot releases a first amount of the therapeutic agent over the first time period and a second amount of the therapeutic agent over the second time period. The first amount can be least 10%, 15%, 20%, 25%, 30%, 35%, 40%, 45%, 50%, 55%, or 60% of the initial amount (e.g., by mass) of the therapeutic agent in the depot; and/or the first amount can be no more than 80%, 75%, 70%, 65%, 60%, 55%, 50%, 45%, 40%, 35%, 30%, or 25% of the initial amount of the therapeutic agent in the depot. The second amount can be at least 5%, 10%, 15%, 20%, 25%, 30%, 35%, 40%, 45%, or 50% of the initial amount of the therapeutic agent in the depot; and/or the second amount can be no more than 60%, 55%, 50%, 45%, 40%, 35%, 30%, 25%, 20%, 15%, or 10% of the initial amount of the therapeutic agent in the depot. Optionally, the depot can release a third amount of the therapeutic agent over a third time period subsequent to the second time period. The third amount can be at least 1%, 2%, 5%, 10%, 15%, 20%, 25%, or 30% of the initial amount of the therapeutic agent in the depot; and/or the third amount can be no more than 40%, 35%, 30%, 25%, 20%, 15%, 10%, or 5% of the initial amount of the therapeutic agent in the depot.

For example, when measured in vitro at pH 5.8, the depot can exhibit the following release profile: the depot can release from 10% to 35% of the therapeutic agent over the first 5 hours to 10 hours of the treatment period; the depot can release from 5% to 65% of the therapeutic agent over the next 25 hours to 35 hours of the treatment period; and/or the depot can release from 1% to 60% of the therapeutic agent over the next 115 hours to 130 hours of the treatment period.

In some embodiments, when measured in vitro at pH 5.8, the depot exhibits the following release profile: the cumulative amount of therapeutic agent released over the first 6 hours to 8 hours of the treatment period is within a range from 5% to 40%, from 10% to 35%, or from 15% to 30% of the initial amount of the therapeutic agent in the depot; the cumulative amount of therapeutic agent released over the first 35 hours to 42 hours of the treatment period is within a range from 35% to 80%, from 37% to 77%, from 40% to 75%, or from 42% to 72% of the initial amount of the therapeutic agent in the depot; and/or the cumulative amount of therapeutic agent released over the first 159 hours to 161 hours of the treatment period is at least 60%, 70%, or 80% of the initial amount of the therapeutic agent in the depot.

In some embodiments, when measured in vitro at pH 5.8, the depot exhibits the following release profile: at least 10% of the therapeutic agent in the depot is released over the first 15 minutes, 30 minutes, 1 hour, 1.5 hours, 2 hours, 2.5 hours, 3 hours, 3.5 hours, 4 hours, 4.5 hours, or 5 hours of the treatment period; at least 20% of the therapeutic agent in the depot is released over the first 1 hour, 1.5 hours, 2 hours, 2.5 hours, 3 hours, 3.5 hours, 4 hours, 4.5 hours, 5 hours, 5.5 hours, or 6 hours of the treatment period; at least 30% of the therapeutic agent in the depot is released over the first 5 hours, 5.5 hours, 6 hours, 6.5 hours, 7 hours, 7.5 hours, 8 hours, 8.5 hours, 9 hours, 9.5 hours, or 10 hours of the treatment period; at least 40% of the therapeutic agent in the depot is released over the first 8 hours, 8.5 hours, 9 hours, 9.5 hours, 10 hours, 10.5 hours, 11 hours, 11.5 hours, 12 hours, 12.5 hours, 13 hours, 13.5 hours, 14 hours, 14.5 hours, or 15 hours of the treatment period; at least 50% of the therapeutic agent in the depot is released over the first 10 hours, 11 hours, 12 hours, 13 hours, 14 hours, 14.5 hours, 15 hours, 15.5 hours, 16 hours, 16.5 hours, 17 hours, 17.5 hours, 18 hours, 18.5 hours, 19 hours, 19.5 hours, or 20 hours of the treatment period; at least 60% of the therapeutic agent in the depot is released over the first 15 hours, 20 hours, 21 hours, 22 hours, 22.5 hours, 23 hours, 23.5 hours, 24 hours, 24.5 hours, 25 hours, 25.5 hours, 26 hours, 26.5 hours, 27 hours, 27.5 hours, 28 hours, 29 hours, or 30 hours of the treatment period; at least 70% of the therapeutic agent is released over the first 30 hours, 31 hours, 32 hours, 33 hours, 34 hours, 35 hours, 36 hours, 37 hours, 38 hours, 39 hours or 40 hours of the treatment period; at least 80% of the therapeutic agent is released over the first 50 hours, 52 hours, 54 hours, 55 hours, 56 hours, 57 hours, 58 hours, 59 hours, 60 hours, 62 hours, 64 hours, or 65 hours of the treatment period; and/or at least 90% of the therapeutic agent in the depot is released over the first 100 hours, 105 hours, 110 hours, 115 hours, 120 hours, 125 hours, 130 hours, 135 hours, 140 hours, 145 hours, or 150 hours of the treatment period.

In some embodiments, when measured in vitro at pH 7.4 and/or in vivo, the depot exhibits the following release profile: the cumulative amount of therapeutic agent released over the first 24 hours of the treatment period is within a range from 1% to 25%, 1% to 10%, or 1% to 5% of the initial amount of the therapeutic agent in the depot; the cumulative amount of therapeutic agent released over the first 48 hours of the treatment period is within a range from 1% to 30%, 5% to 20%, or 5% to 15% of the initial amount of the therapeutic agent in the depot; the cumulative amount of therapeutic agent released over the first 72 hours of the treatment period is within a range from 1% to 5%, 2% to 15%, 10% to 35%, 10% to 25%, or 15% to 25% of the initial amount of the therapeutic agent in the depot; the cumulative amount of therapeutic agent released over the first 96 hours of the treatment period is within a range from 15% to 50%, 10% to 40%, or 10% to 30% of the initial amount of the therapeutic agent in the depot; the cumulative amount of therapeutic agent released over the first 120 hours of the treatment period is within a range from 20% to 60%, 25% to 50%, or 30% to 40% of the initial amount of the therapeutic agent in the depot; the cumulative amount of therapeutic agent released over the first 144 hours of the treatment period is within a range from 25% to 70%, 30% to 50%, or 35% to 45% of the initial amount of the therapeutic agent in the depot; the cumulative amount of therapeutic agent released over the first 7 days to 8 days of the treatment period is within a range from 30% to 70%, or 35% to 55% of the initial amount of the therapeutic agent in the depot; the cumulative amount of therapeutic agent released over the first 14 days of the treatment period is within a range from 50% to 90%, or 60% to 80% of the initial amount of the therapeutic agent in the depot; and/or the cumulative amount of therapeutic agent released over the first 21 days of the treatment period is within a range from 70% to 99%, or 85% to 95% of the initial amount of the therapeutic agent in the depot.

In some embodiments, when measured in vitro at pH 7.4 and/or in vivo, the depot exhibits the following release profile: up to 10% of the therapeutic agent in the depot is released over the first 4 hours, 12 hours, 24 hours, or 48 hours of the treatment period; up to 20% of the therapeutic agent in the depot is released over the first 24 hours, 48 hours, 72 hours, or 84 hours of the treatment period; up to 30% of the therapeutic agent in the depot is released over the first 48 hours, 72 hours, 120 hours, or 144 hours of the treatment period; up to 40% of the therapeutic agent in the depot is released over the first 120 hours, 144 hours, 168 hours, or 192 hours of the treatment period; up to 50% of the therapeutic agent in the depot is released over the first 7 days, 8 days, 9 days, or 10 days of the treatment period; up to 60% of the therapeutic agent in the depot is released over the first 10 days, 11 days, 12 days, or 13 days of the treatment period; up to 70% of the therapeutic agent in the depot is released over the first 13 days, 14 days, 15 days, or 16 days of the treatment period; up to 80% of the therapeutic agent in the depot is released over the first 16 days, 17 days, 18 days, or 19 days of the treatment period; and/or up to 90% of the therapeutic agent in the depot is released over the first 19 days, 20 days, 21 days, or 22 days of the treatment period.

In some embodiments, the release profile of the therapeutic agent is a first order release profile (which can be modeled by the equation Qₜ=Q₀e^{-kt} where Qₜ is the amount of therapeutic agent released at time t, Q₀ is the initial amount of therapeutic agent in the depot, and k is the rate constant). Alternatively, the release profile can be a zero order release profile, a second order release profile, or any other suitable release profile known to those of skill in the art.

The depots described herein can be configured to release a larger amount of the therapeutic agent per day for a first time period than for a longer second time period. In some embodiments, the depot is configured to release the therapeutic agent for at least 14 days post-implantation (or post-immersion in a fluid), where a controlled burst of about 20% to about 50% of the therapeutic agent payload is released in the first 3 days to 5 days, and at least 80% of the remaining therapeutic agent payload is released at a slower rate over the last 10 days to 11 days. In some embodiments, at least 90% of the therapeutic agent payload is released by the end of 14 days.

A two-stage release profile may be especially beneficial in the context of treating pain resulting from a total knee arthroplasty (“TKA”). TKA patients typically experience the greatest pain within the first 1 day to 3 days following surgery (clinically referred to as “acute pain”) with increasingly less pain over the next 7 days to 10 days (clinically referred to as “subacute pain”). The acute period often overlaps or coincides with the patient's inpatient care (usually 1 day to 3 days), and the subacute period generally begins when the patient is discharged and returns home. The two-stage release profile can also be beneficial for other surgical applications, such as other orthopedic applications (e.g., ligament repair/replacement and other damage to the knee, shoulder, ankle, etc.) or non-orthopedic surgical applications, as described in greater detail below. Excessive pain following any surgery may extend inpatient care, cause psychological distress, increase opioid consumption, and/or impair patient participation in physical therapy, any of which may prolong the patient's recovery and/or mitigate the extent of recovery. Pain relief during the subacute period may be particularly complicated to manage, as patient compliance with the prescribed pain management regimen drops off when patients transition from an inpatient to home environment.

To address the foregoing challenges in post-surgical pain management, the depots of the present technology may have a release profile tailored to meet the pain management needs specific to the acute and subacute periods. For example, to address the greater acute pain that occurs immediately following surgery, the depot can be configured to release the therapeutic agent at a faster rate for the first 3 days to 5 days after implantation compared to the subsequent 9 days to 11 days. In some embodiments, the depot delivers a local anesthetic at a rate from about 150 mg/day to about 400 mg/day during this first, acute period. To address the diminishing pain during the subacute period, the depot can be configured to release the therapeutic agent at a slower rate for the remaining 9 days to 11 days. In some embodiments, the depot delivers a local anesthetic at a rate from about 50 mg/day to about 250 mg/day during this second, subacute period. In some embodiments, the rate of release continuously decreases throughout the first period and/or the second period.

The release profile of the depot can be tuned to release a therapeutic agent for other durations and/or at other release rates by adjusting the structure, composition, and/or the process by which the depot is manufactured. For example, in some embodiments, the depot is configured to release the therapeutic agent at a constant rate throughout the entire duration of release. In some embodiments, the depot is to release the therapeutic agent at a constant rate for a first time period and at a non-constant rate for a second time period (which may occur before or after the first time period).

In some embodiments, the depot is configured to release no more than 20%, 25%, 30%, 35%, 40%, 45%, 50%, 55%, 60%, 65%, or 70% of the therapeutic agent in the first day, 2 days, 3 days, 4 days, 5 days, 6 days, 8 days, 9 days, 10 days, 11 days, 12 days, or 13 days of the duration of release, and at least 75%, 80%, 85%, 90%, 95%, or 100% of the remaining therapeutic agent is released in the remaining days of the duration of release. The intended duration of release may be at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 40 days, 50 days, 60 days, 70 days, 80 days, 90 days, 100 days, or 120 days.

In some embodiments, the depot is configured to release from 50 mg/day to 600 mg/day, from 100 mg/day to 500 mg/day, from 100 mg/day to 400 mg/day, or from about 100 mg/day to 300 mg/day of the therapeutic agent to the treatment site. In general, the release rate can be selected to deliver the desired dosage to provide the extent of pain relief needed at a given time after the surgical procedure, control toxicity, and deliver the therapeutic agent for a sufficient period of time for pain relief. In some embodiments, the depot is configured to release 50 mg, 100 mg, 150 mg, 200 mg, 250 mg, 300 mg, 350 mg, 400 mg, 450 mg, 500 mg, 600 mg, 700 mg, 800 mg, 900 mg, or 1000 mg of therapeutic agent within any day of the duration of release.

In some embodiments, the depot is configured to release from 50 mg/day to 600 mg/day, from 100 mg/day to 500 mg/day, from 100 mg/day to 400 mg/day, or from 100 mg/day to 300 mg/day of the therapeutic agent to the treatment site within a first time period of release. The depot can further be configured to release from 500 mg/day to 600 mg/day, from 100 mg/day to 500 mg/day, from 100 mg/day to 400 mg/day, or from 100 mg/day to 300 mg/day of the therapeutic agent to the treatment site within a second time period of release. The release rate during the first time period can be the same as, different than, less than, or greater than the release rate during the second time period. Moreover, the first time period can be longer or shorter than the second time period. The first time period can occur before or after the second time period.

In some embodiments, the depot is configured to release no more than 50 mg, 100 mg, 150 mg, 200 mg, 250 mg, 300 mg, 350 mg, 400 mg, 450 mg, 500 mg, 600 mg, 700 mg, 800 mg, 900 mg, 1000 mg of the therapeutic agent within any day of a first time period of release. Alternatively or in combination, the depot can be configured to release at least 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, or 300 mg of the therapeutic agent within any day of the first time period of release. This may be useful for providing different degrees of pain relief at different times after the surgical procedure, and it may also be useful to control toxicity. In such embodiments, the depot can be configured to release at least 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 350 mg, 400 mg, 450 mg, 500 mg, 600 mg, 700 mg, 800 mg, 900 mg, or 1000 mg of the therapeutic agent within any day of a second time period of release. The first time period and/or the second time period can be 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days.

One or more depots of the present technology can be implanted at a treatment site in order to produce a desired level of therapeutic agent in vivo, such as a level at or above a therapeutic threshold and/or below a toxicity threshold. For example, when implanted, one or more depots of the present technology can produce a mean plasma concentration of the therapeutic agent greater than or equal to a therapeutic threshold of 5 ng/ml, 10 ng ml, 15 ng/ml, 20 mg/ml, 25 ng/ml, 30 ng/ml, 40 ng/ml, 50 ng/ml, 60 ng/ml, 70 ng/ml, 80 ng/ml, 90 ng/ml, 100 ng/ml, 110 ng/ml, 120 ng/ml, 130 ng/ml, 140 ng/ml, 150 ng/ml, 160 ng/ml, 170 ng/ml, 180 ng/ml, 190 ng/ml, 200 ng/ml, 210 ng/ml, 220 ng/ml, 230 ng/ml, 240 ng/ml, 250 ng/ml, 300 ng/ml, 400 ng/ml, 500 ng/ml, 600 ng/ml, 700 ng/ml, 800 ng/ml, 900 ng/ml, or 1000 ng/ml. Alternatively or combination, the depot(s) can produce a mean plasma concentration of the therapeutic agent less than or equal to a toxicity threshold of 9000 ng/ml, 8000 ng/ml, 7000 ng/ml, 6000 ng/ml, 5000 ng/ml, 4000 ng/ml, 3000 ng/ml, 2500 ng/ml, 2400 ng/ml, 2300 ng/ml, 2200 ng/ml, 2100 ng/ml, 2000 ng/ml, 1900 ng/ml, 1800 ng/ml, 1700 ng/ml, 1600 ng/ml, 1500 ng/ml, 1400 ng/ml, 1300 ng/ml, 1200 ng/ml, 1100 ng/ml, or 1000 ng/ml. The mean plasma concentration of the therapeutic agent can be maintained above the therapeutic threshold and/or below the toxicity threshold for at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 40 days, 50 days, 60 days, 70 days, 90 days, 100 days, 200 days, 300 days, or 365 days.

In some embodiments, when implanted, the depot(s) produce a mean Cₘₐₓ of the therapeutic agent that is less than or equal to 1000 ng/ml, 900 ng/ml, 800 ng/ml, 700 ng/ml, 600 ng/ml, 500 ng/ml, 400 ng/ml, 300 ng/ml, 200 ng/ml, 100 ng/ml, or 50 ng/ml. The depot(s) can produce a mean tin of the therapeutic agent that is greater than or equal to 1 day, 2 days, 3 days, 4 days, 5 days, 6 days, or 7 days. The depot(s) can produce a mean tₘₐₓ of the therapeutic agent that is at least 1 hour, 2 hours, 4 hours, 12 hours, 24 hours, 48 hours, 36 hours, 72 hours, 96 hours, 120 hours, 144 hours, or 168 hours. The depot(s) can produce a mean tlast of the therapeutic agent that is at least 7 days, 8 days, 9 days, 10 days, 12 days, 13 days, 14 days, 15 days, 16 days, 20 days, 25 days, 30 days, 35 days, 40 days, or 45 days.

In some embodiments, when implanted, the depot(s) produce a mean AUCₜ₁₋ₜ₂ of the therapeutic agent that is at least 500 day-ng/ml, 1000 day-ng/ml, 1500 day-ng/ml, 2000 day-ng/ml, 2500 day-ng/ml, 3000 day-ng/ml, 3500 day-ng/ml, 4000 day-ng/ml, 4500 day-ng/ml, 5000 day-ng/ml, 5500 day-ng/ml, 6000 day-ng/ml, 6500 day-ng/ml, 7000 day-ng/ml, 7500 day-ng/ml, or 8000 day-ng/ml; where the time period t1-t2 can be any of the following: 0 days to 7 days, 0 days t0 14 days, 0 days to 21 days, 0 days to 30 days, 3 days to 7 days, 7 days to 14 days, 7 days to 21 days, 7 days to 30 days, 14 days to 21 days, 14 days to 30 days, or 21 days to 30 days. The depot(s) can produce a mean AUCₗₐₛₜ of the therapeutic agent that is at least 500 day-ng/ml, 1000 day-ng/ml, 1500 day-ng/ml, 2000 day-ng/ml, 2500 day-ng/ml, 3000 day-ng/ml, 3500 day-ng/ml, 4000 day-ng/ml, 4500 day-ng/ml, 5000 day-ng/ml, 5500 day-ng/ml, 6000 day-ng/ml, 6500 day-ng/ml, 7000 day-ng/ml, 7500 day-ng/ml, or 8000 day-ng/ml.

In some embodiments, the therapeutic agent is configured to elute from the implantable depot at a sufficiently slow rate so that the depot maintains sufficient flexural strength and/or mechanical integrity in vivo for at least a predetermined period of time or until a predetermined proportion of therapeutic agent has been released from the depot. The depot can be considered to maintain its structural integrity if the depot remains largely intact with only partial or gradual reduction due to elution of therapeutic agent or dissolution of the releasing agent. The depot can be considered to lose its structural integrity if it separates (e.g., fractures) into multiple component pieces, for example, with two or more of the resulting pieces being at least 5% of the previous size of the depot. Alternatively, or additionally, the depot can be considered to lose its structural integrity if the release rate of the therapeutic agent increases by more than a factor of three as compared to the release rate of therapeutic agent in a control depot submerged in a buffered solution.

In some embodiments, the depot is configured to maintain its structural integrity in vivo for at least a predetermined length of time. For example, the depot can be configured to maintain its structural integrity in vivo for at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 40 days, 50 days, 60 days, 70 days, 90 days, 100 days, 200 days, 300 days, or 365 days. In some embodiments, the depot is configured to maintain its structural integrity in vivo until at least a predetermined proportion of therapeutic agent payload has been released from the depot. For example, the depot can be configured to maintain its structural integrity in vivo until at least 5%, 10%, 15%, 20%, 25%, 30%, 35%, 40%, 45%, 50%, 55%, 60%, 65%, 70%, 75%, 80%, 85%, 90%, 95%, or 100% of the original mass of the therapeutic agent in the depot has been released.

### II. Systems and Methods of Use

The implantable depots of the present technology can be used to treat a variety of injuries, conditions, or diseases, depending upon the nature of the therapeutic agent delivered as described above. The therapeutic agent can be delivered to specific areas of the patient's body depending upon the medical condition being treated. The depots of the present technology can be positioned in vivo proximate to the target tissue (e.g., bone, soft tissue, nerve, etc.) in the patient's body to provide a controlled, sustained release of a therapeutic agent for the treatment of a particular condition. This implantation can be associated with a surgery or intervention for acutely treating the particular condition, whereby the depot provides chronic, sustained pharmacological treatment following completion of the surgery or intervention. The depot can be a standalone element, or can be coupled to or integrated as part of an implantable device or prosthesis associated with the intervention or surgery.

The amount or dose of the therapeutic agent that will be effective in a patient in need thereof can depend on the specific nature of the condition, and can be determined by standard clinical techniques known in the art. In addition, in vitro or in vivo assays can optionally be employed to help identify optimal dosage ranges. The specific dose level for any particular individual will depend upon a variety of factors including the activity of the drug, the age, body weight, general physical and mental health, genetic factors, environmental influences, sex, diet, time of administration, location of administration, rate of excretion, and/or the severity of the particular problem being treated.

Some aspects of the present technology include a system including one or more depots (each of which could be any of the depots described herein) provided for implantation by a clinical practitioner. For example, a system can include one, two, three, four, five, six, seven, eight, nine, ten, or more implanted depots. Each depot can be configured for controlled release of a therapeutic agent to tissue proximate to the implantation site of the depot. Accordingly, the depots can collectively provide a desired dose of the therapeutic agent, such as a dose greater than or equal to 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1100 mg, 1200 mg, 1300 mg, 1400 mg, 1500 mg, 1600 mg, 1700 mg, or 1800 mg. The dose provided by an individual depot or a set of depots can be expressed in terms of the mass of the therapeutic agent used in the depot(s), or in terms of the mass of another form of the therapeutic agent (e.g., the form of the active moiety or the established salt form). For example, the dose of bupivacaine in a depot formulated with bupivacaine hydrochloride monohydrate may be expressed in terms of the equivalent mass of bupivacaine free base (e.g., 594 mg of bupivacaine hydrochloride monohydrate is equivalent to 500 mg of bupivacaine free base) or in terms of the equivalent mass of bupivacaine hydrochloride (e.g., 595 mg bupivacaine hydrochloride monohydrate is equivalent to 564 mg of bupivacaine hydrochloride).

In embodiments where the depot is configured as a coating on a medical device, the dose provided by the depot can be expressed as in terms of a dose density, e.g., mass of the therapeutic agent per surface area of the coating. For example, the coating can include a dose density within a range from 0.1 mg/mm² to 10 mg/mm², 0.1 mg/mm² to 5 mg/mm², 0.1 mg/mm² to 1 mg/mm², 0.1 mg/mm² to 0.5 mg/mm², 0.5 mg/mm² to 10 mg/mm², 0.5 mg/mm² to 5 mg/mm², 0.5 mg/mm² to 1 mg/mm², 1 mg/mm² to 10 mg/mm², 1 mg/mm² to 5 mg/mm², or 5 mg/mm² to 10 mg/mm².

In embodiments where the system includes multiple depots, some or all of the depots in the system can be identical and/or some or all of the depots can differ from each other (e.g., with respect to geometry, composition, and/or release profile). For example, the system can include at least one depot having a release profile that provides for an immediate release of a therapeutic agent, and at least one other depot having a release profile that provides for a delayed release of the therapeutic agent.

Many depots of the present technology are configured to be implanted at a surgical site to treat postoperative pain at or near the site. As used herein, the term “pain” includes nociception and the sensation of pain, both of which can be assessed objectively and subjectively, using pain scores and other methods well-known in the art, such as opioid usage, as described in further detail below. Pain can include allodynia (e.g., increased response to a normally non-noxious stimulus) or hyperalgesia (e.g., increased response to a normally noxious or unpleasant stimulus), which can in turn be thermal or mechanical (tactile) in nature. In some embodiments, pain is characterized by thermal sensitivity, mechanical sensitivity, and/or resting pain. The pain can be primary or secondary pain, as is well-known in the art. Exemplary types of pain reducible, preventable or treatable by the methods and compositions disclosed herein include, without limitation, postoperative pain, for example, from the back in the lumbar regions (lower back pain) or cervical region (neck pain), leg pain, radicular pain (experienced in the lower back and leg from lumbar surgery in the neck and arm from cervical surgery), or abdominal pain from abdominal surgery, and neuropathic pain of the arm, neck, back, lower back, leg, and related pain distributions resulting from disk or spine surgery. Neuropathic pain may include pain arising from surgery to the nerve root, dorsal root ganglion, or peripheral nerve.

In some embodiments, the pain includes “post-surgical pain,” “postoperative pain,” or “surgery-induced pain,” which are used herein interchangeably, and refer to pain arising in the recovery period of seconds, minutes, hours, days or weeks following a surgical procedure (e.g., hernia repair, orthopedic or spine surgery, etc.). Surgical procedures can include any procedure that penetrates beneath the skin and causes pain and/or inflammation to the patient. Surgical procedures can be performed at various sites in a patient's body. For example, surgery may be performed at a patient's knees, hips, upper extremities, lower extremities, neck, spine, shoulders, chest, nasal/sinus region, abdomen, and/or pelvic region.

Some embodiments of the present technology include one or more depots (e.g., having the same or different configuration and/or dosing) that are positioned at or near a surgical site of a knee joint to treat pain associated with a total knee replacement surgery, also known as TKA. In some instances, it may be beneficial to position one or more of the depots within the joint capsule. In some embodiments, one or more depots are positioned at or near the suprapatellar pouch, specifically under the periosteum and attached to the quadriceps tendon. Additional areas for placement of one or more depots may include generally the medial and lateral gutters (including optional fixation to tissue at the medial or lateral side of the respective gutter), on the femur, on the tibia (e.g., posterior attachment to the tibial plateau, at or near the anterior tibia to anesthetize infrapatellar branches of the saphenous nerve). In some embodiments, one or more depots are positioned adjacent to at least one of a posterior capsule of the knee, a superior region of the patella, and/or the arthrotomy incision into the knee capsule. In some embodiments, one or more depots are positioned at or near the saphenous nerve, the adductor canal, and/or the femoral nerve. In some embodiments, one or more depots are positioned at or near an infrapatellar branch of the saphenous nerve, one or more genicular nerves of the knee, a superior region of the patella. It may be desirable to position the depot(s) within the knee capsule but away from any articulating portions of the knee joint itself.

In some embodiments, one or more depots are positioned at or near one or more nerves innervating an anterior knee capsule. For example, the depot(s) may be configured to be positioned at or near a superolateral genicular branch from the vastus lateralis, a superomedial genicular branch from the vastus medialis, a medial (retinacular) genicular branch from the vastus intermedius, an inferolateral genicular branch from the common peroneal nerve, an inferomedial genicular branch from the saphenous nerve, and/or a lateral (retinacular) genicular branch from the common peroneal nerve. Instead of or in addition to the placement of depots within the intracapsular space, one or more depots may be placed at an extracapsular position. In some embodiments, the depot(s) are implanted adjacent to one or more extracapsular nerves. In some embodiments, one or more depots are positioned along or adjacent the subcutaneous skin incision.

So as not to interfere or overlap with a peripheral nerve block administered perioperatively to the patient, one or more of the depots may optionally include a delayed release capability for 6 hours to 24 hours following implantation. In some embodiments, one or more depots placed in the adductor canal and knee capsule are configured to have a delay in the release of therapeutic agent of at least 24 hours.

In some embodiments, the depots of the present technology utilize regional procedures for controlling pain following TKA. Such procedures can include local anesthetic infiltration between the popliteal artery and capsule of the knee (IPACK) block. An IPACK block procedure typically involves scanning the popliteal fossa using a probe proximal the popliteal crease, and injecting an analgesic (e.g., 20 ml of 0.25% ropivacaine) between the patient's popliteal artery and femur. Unlike other known procedures (e.g., adductor canal block (ACB) and femoral nerve catheter (FNC) block) for treating postoperative pain following TKA, IPACK block targets only the terminal branches of the sciatic nerve. In doing so, analgesia and/or other therapeutic agents can be provided to the posterior knee region without causing distal neurologic deficits. In some embodiments, the depots of the present technology are implanted using a combination of the IPACK block procedure and the ACB or FNC block procedures. For example, patients can preoperatively receive one or more depots utilizing an FNC block, and then receive one or more additional depots utilizing a postoperative IPACK block. Utilizing the IPACK block procedure with depots of the present technology can advantageously provide adequate analgesia following TKA, promote improved physical therapy performance, reduce the incident of foot drop, reduce opioid consumption, and/or better control posterior knee pain following TKA, e.g., relative to ACB, FNC block, or other known techniques for pain management following TKA, often allowing for earlier hospital discharge.

The depots disclosed herein can be used to treat postoperative pain associated with other knee surgeries. For example, one or more depots may be used to treat postoperative pain associated with an ACL repair surgery, a medial collateral ligament (“MCL”) surgery, and/or a posterior cruciate ligament (“PCL”) surgery. For ACL repair, one or more depots may be positioned to deliver analgesic to the femoral and/or sciatic nerves, while for PCL repair surgery, one or more depots may be positioned parasacral to deliver analgesic to the sciatic nerve. The one or more depots may be used to treat postoperative pain associated with a partial knee replacement surgery, total knee replacement surgery, and/or a revision surgery of a knee replacement surgery. In such procedures, one or more depots can be placed contiguous to the joint or repair site to provide a local block, or else may suitably positioned to provide a regional block by delivering an analgesic to one or more of the femoral nerve or the sciatic nerve, for example via placement in the adductor canal.

In addition to the knee-related surgeries described above, embodiments of the depots disclosed herein can be used to treat postoperative pain associated with other orthopedic surgeries, such as procedures involving the ankle, hip, shoulder, wrist, hand, spine, legs, or arms. For at least some of these surgical procedures, an analgesic can be provided to deliver a local block or a regional block to treat postoperative pain. For a local block, one or more depots can be positioned under direct vision in open surgery, for example during joint arthroplasty, open reduction and internal fixation (ORIF) surgery, ligament reconstruction, etc. In procedures involving a joint, one or more depots can be positioned at the joint capsule (e.g., at or near the intracapsular and/or extracapsular space of the joint) and/or adjacent soft tissues spaced apart from articulating surfaces to avoid the depot interfering with joint movement or being damaged by contact with articulating surfaces. In procedures involving fracture repair or ligament repair, one or more depots can be positioned at or adjacent to the repair site to provide a local block. For a regional block, one or more depots can be deposited at a treatment site adjacent to the target nerve via ultrasound guidance using a blunt trocar catheter or other suitable instrument. In some embodiments, it can be beneficial to combine delivery of an analgesic or other therapeutic agents via the depot(s) with delivery of NSAIDs, a long-acting narcotic delivered pre-operatively, and/or acetaminophen. The sustained, controlled, release of an analgesic via the one or more depots can work in concert with these other therapeutic agents to provide a reduction in postoperative pain associated with orthopedic and other surgical procedures.

For example, one or more depots can be used to treat postoperative pain associated with foot and ankle surgeries, such as ankle arthroplasty (including ankle revision, ankle replacement, and total ankles replacement), ankle fusion, ligament reconstruction, corrective osteotomies (e.g., bunionectomy, pes planus surgery), or ORIF of ankle or foot fractures. In treating postoperative pain associated with such surgeries, one or more depots can be configured and positioned adjacent to the joint or repair site to provide a local block. Additionally or alternatively, one or more depots can be placed parasacral or at another suitable location to target one or more of the subgluteal sciatic nerve, popliteal sciatic nerve, deep peroneal nerve, or the superficial peroneal nerve. In some embodiments, depots positioned to treat postoperative pain associated with ankle or foot surgeries have a release profile configured to deliver therapeutically beneficial levels of analgesic for a period of 3 days to 7 days.

In another example, one or more depots can be used to treat postoperative pain associated with hip surgeries, such as hip arthroplasty (including hip revision, partial hip replacement, and total hip replacement) or ORIF of hip fractures. In treating postoperative pain associated with such surgeries, one or more depots can be configured and positioned adjacent to the joint or repair site to provide a local block. Additionally or alternatively, a regional block can be provided by placing depots in the psoas compartment, lumbar paravertebral space, fascia iliaca, or other suitable location to target one or more of the lumbar plexus, sacral plexus, femoral nerve, sciatic nerve, superior gluteal nerve, or obturator nerve. In some embodiments, it may be beneficial to secure the one or more depot(s) (e.g., using sutures, fasteners, or other fixation mechanisms) to maintain an anterior position of the depot, thereby preventing or reducing exposure of analgesic to motor nerves (e.g., sciatic or femoral nerves). In some embodiments, depots positioned to treat postoperative pain associated with hip surgeries have a release profile configured to deliver therapeutically beneficial levels of analgesic for a period of 5 days to 7 days, or 7 days to 10 days, depending on the particular surgical procedure.

Postoperative pain associated with shoulder and upper-arm surgeries can likewise be treated using one or more depots as disclosed herein. Examples of such surgeries include shoulder arthroplasty (including shoulder revision, partial shoulder replacement, and total shoulder replacement), upper-arm fracture repair (e.g., scapular, humerus), ligament/tendon repair (e.g., rotator cuff, labrum, biceps, etc.), or ORIF of fractures of the shoulder or upper arm. In treating postoperative pain associated with such surgeries, one or more depots can be configured and positioned adjacent to the joint or repair site to provide a local block. Additionally or alternatively, one or more depots can be configured and positioned to target the brachial plexus by placing one or more depots in the cervical paravertebral space, interscalene, or supraclavicular space. In some embodiments, interscalene placement of the depots can avoid exposure of analgesic to native cartilage, thereby reducing the risk of chondrotoxicity. In some embodiments, depots positioned to treat postoperative pain associated with shoulder or upper-arm related surgeries have a release profile configured to deliver therapeutically beneficial levels of analgesic for a period of 3 days to 7 days.

In another example, one or more depots as described herein can be used to treat postoperative pain associated with elbow surgeries, such as elbow arthroplasty (including elbow revision, partial elbow replacement, and total elbow replacement), ligament reconstruction, or ORIF of fractures of the elbow. In treating postoperative pain associated with such surgeries, one or more depots can be positioned adjacent to the joint or repair site to provide a local block. Additionally or alternatively, one or more depots can be configured and positioned to target the brachial plexus nerves, for example by being placed at or near the cervical paravertebral space, infraclavicular, or axillary position, or other suitable location. In some embodiments, depots positioned to treat postoperative pain associated with elbow surgeries have a release profile configured to deliver therapeutically beneficial levels of analgesic for a period of 3 days to 7 days.

Postoperative pain associated with wrist and hand surgeries can also be treated using one or more depots as described herein. Examples of wrist and hand surgeries include wrist arthroplasty (including wrist revision, partial wrist replacement, and total wrist replacement), wrist fusion, and ORIF of fractures of the wrist. In treating postoperative pain associated with such surgeries, one or more depots can be configured and positioned adjacent to the wrist joint or repair site to provide a local block. Additionally or alternatively, one or more depots can be configured and positioned to target the target the ulnar, median, radial, and cutaneous forearm nerves, for example via placement at the antecubital fossa, cervical paravertebral space, infraclavicular, or axillary position. In some embodiments, depots positioned to treat postoperative pain associated with wrist and hand surgeries have a release profile configured to deliver therapeutically beneficial levels of analgesic for a period of 3 days to 7 days.

The depots disclosed herein can likewise be used to treat postoperative pain from other orthopedic surgeries. For example, postoperative pain associated with spinal fusion can be treated via placement of one or more depots subcutaneously or in the paravertebral space. In treatment of postoperative pain associated with fibular fracture repair, one or more depots can be configured and placed to target the sciatic nerve and/or the popliteal sciatic nerve, for example, being placed parasacral. Various other placements and configurations are possible to provide therapeutic relief from postoperative pain associated with orthopedic surgical procedures.

The depots disclosed herein may be used to treat postoperative pain associated with other types of surgeries besides orthopedic surgeries. For example, the depots may be used to treat postoperative pain for chest-related surgery; breast-related surgery; gynecological or obstetric surgery; general surgery, abdominal surgery; urological surgery; ear, nose, and throat (ENT) surgery; oral and maxillofacial surgery; oncological surgery; reconstructive surgery; or cosmetic surgery. For particular surgeries or classes of surgeries, one or more depots can be positioned at a treatment site to treat postoperative pain. The treatment site can be at or near the surgical site, or can be spaced apart from the surgical site (e.g., proximate to a target nerve or nerve bundle that innervates the surgical site).

For example, one or more depots as described herein can be used to treat postoperative pain associated with chest-related surgeries, such as a thoracotomy, sternotomy, Nuss procedure, esophageal surgery, cardiac surgery, lung resection, thoracic surgery, or other such procedure. In treating postoperative pain associated with such surgeries, one or more depots can be configured and positioned to target the intercostal nerves, for example, by being placed at or near the thoracic paravertebral space or other suitable location. Analgesics delivered to the intercostal nerves can reduce pain in a patient's chest area, thereby relieving postoperative pain associated with the above-noted chest-related surgical procedures.

In another example, one or more depots disclosed herein can be used to treat postoperative pain associated with breast-related surgeries, such as a mastectomy, breast augmentation (mammoplasty), breast reduction, breast reconstruction procedure, or other such procedures. To treat postoperative pain from such procedures, one or more depots can be positioned and configured to deliver analgesics or other therapeutic agents to the intercostal nerves, for example via placement at or near the patient's infraclavicular space or other suitable location. Additionally or alternatively, one or more depots can be positioned and configured to deliver analgesics or other therapeutic agents to the lateral pectoral nerve and/or the medial pectoral nerve, for example, via placement between the serratus anterior muscle and the latissimus dorsi muscle or other suitable location. As noted above, analgesics delivered to the intercostal nerves can reduce pain in a patient's chest area, while analgesics delivered to the lateral and/or medial pectoral nerves can reduce pain in the pectoralis major and pectoralis minor, thereby reducing postoperative pain associated with the above-noted chest-related surgical procedures.

As another example, one or more depots can be used to treat postoperative pain associated with general, abdominal, and/or urological procedures. Examples of such procedures include proctocolectomy, pancreatectomy, appendectomy, hemorrhoidectomy, cholecystectomy, kidney transplant, nephrectomy, radical prostatectomy, nephrectomy, gastrectomy, small bowel resection, splenectomy, incisional hernia repair, inguinal hernia repair, ventral hernia repair, sigmoidectomy, colorectal resection, liver resection, enterostomy, rectum resection, kidney stone removal, and cystectomy procedures. For such operations, postoperative pain can be treated by placing one or more depots to target nerves at the transverse abdominis plane (TAP). Analgesics delivered to the TAP can anesthetize the nerves that supply the anterior abdominal wall, thereby reducing postoperative pain in this region. In some embodiments, one or more depots are disposed between the internal oblique and transverse abdominis muscles. In some embodiments, one or more depots can be disposed at or adjacent to the abdominal wall, for example, being secured in place via sutures, fasteners, or other fixation mechanisms. Other locations that can be targeted include the rectus sheath, ilioinguinal nerve, and/or iliohypogastric nerve.

In some embodiments, one or more depots are used to treat postoperative pain associated with gynecological and obstetric surgeries, such as myomectomy, Caesarian section, hysterectomy, oophorectomy, pelvic floor reconstruction, or other such surgical procedures. For such procedures, the depot(s) can be configured and positioned to deliver analgesics or other therapeutic agents to one or more of the nerves innervating the pelvic and/or genital area, for example, the pudendal nerve, paracervical nerve, intercostal nerve, or other suitable nerve.

In some embodiments, one or more depots can be used to treat postoperative pain associated with ENT surgical procedures, for example, tonsillectomy, submucosal resection, rhinoplasty, sinus surgery, inner ear surgery, parotidectomy, submandibular gland surgery, tympanostomy, exostosis surgery (surfer's ear surgery), or other such procedures. Similarly, one or more depots can be used to treat postoperative pain associated with oral and maxillofacial surgeries, for example, dentoalveolar surgery, dental implant surgery, orthognathic surgery, temporomandibular joint (TMJ) surgery, dental reconstruction surgeries, or other such procedures. For ENT surgical procedures and/or oral and maxillofacial surgical procedures, the depot(s) can be configured and positioned to deliver analgesics or other therapeutic agents to one or more of the nerves innervating regions affected by the surgical procedure, for example, the mandibular nerve, the mylohyoid nerve, lingual nerve, inferior alveolar nerve, buccal nerve, auriculotemporal nerve, anterior ethmoidal nerve, or other suitable nerve.

One or more depots can also be used to treat postoperative pain for other surgical procedures, for example oncological surgeries (e.g., tumor resection), cosmetic surgeries (e.g., liposuction, abdominoplasty), or other surgical procedures resulting in postoperative pain. For treatment of postoperative pain associated with any particular surgery, the number of depots and the characteristics of individual depots (e.g., geometry, composition, release profile) can be selected to deliver the desired therapeutic benefits. For example, while a patient recovering from a knee replacement surgery may benefit from delivery of analgesics for at least 14 days, a patient recovering from a tonsillectomy may not require the same level or duration of analgesic drug delivery. As such, depots delivered to a patient for treatment of postoperative pain following a tonsillectomy may require fewer depots, or depots having a smaller payload of therapeutic agent, or depot(s) having a faster release profile, etc. Additionally, the number and characteristics of the depot(s) selected for implantation can be tailored to accommodate the target anatomical region for placement in the patient's body.

In some embodiments, the depots of the present technology are used to treat other types of pain indications, such as chronic pain, pain resulting from a disease or condition, or pain resulting from a wound. Examples of pain indications that can be treated with the depots herein include shingles pain, phantom limb pain, arthritis pain (e.g., osteoarthritis pain), frozen shoulder pain, chronic back pain, sciatica pain, bone fracture pain (e.g., rib fractures, collarbone fracture, toe fracture, and/or other fractures that do not require surgery), pain resulting from chronic cough (e.g., abdominal and/or chest pain due to strain from coughing), wound pain (e.g., burns, diabetic lesions, bed sores), and cancer pain (e.g., pain due to cancer and/or cancer therapy (e.g., chemotherapy, radiotherapy)). For instance, one or more depots of the present technology can be placed proximate to or upstream of one or more nerves innervating the site of the pain, thus acting as a nerve block. Depots including a hydrophobic therapeutic agent (e.g., the hydrophobic free base form of an amine-containing analgesic) can provide sustained release over longer time periods, and thus may be less invasive for management of chronic pain and other long-term pain indications than continuous infusion.

### EXAMPLES

The following examples are included to further describe some aspects of the present technology, and should not be used to limit the scope of the technology.

### Example 1: Manufacturing Bupivacaine Free Base Depots

Implantable depots configured in accordance with embodiments of the present technology were manufactured using a melt form process. Each depot was composed of 100% bupivacaine free base (Cambrex) by mass.

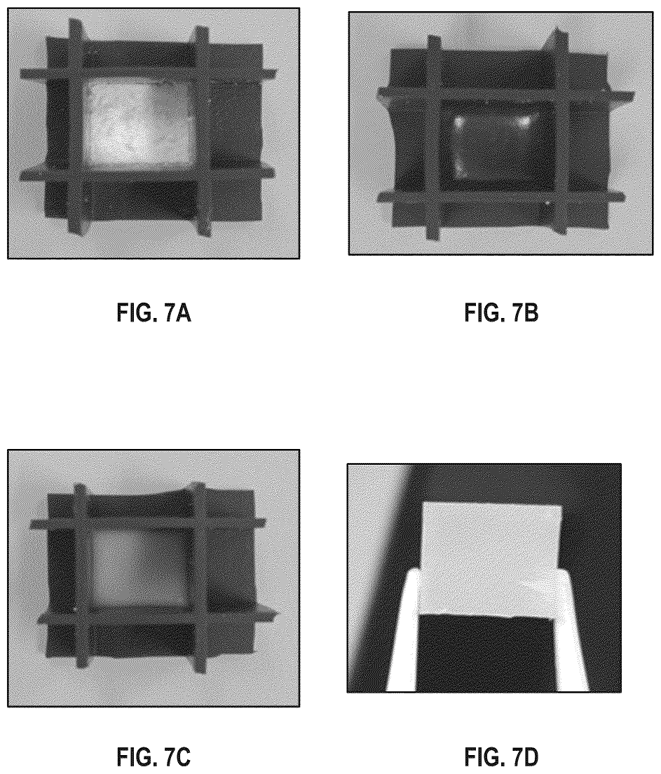

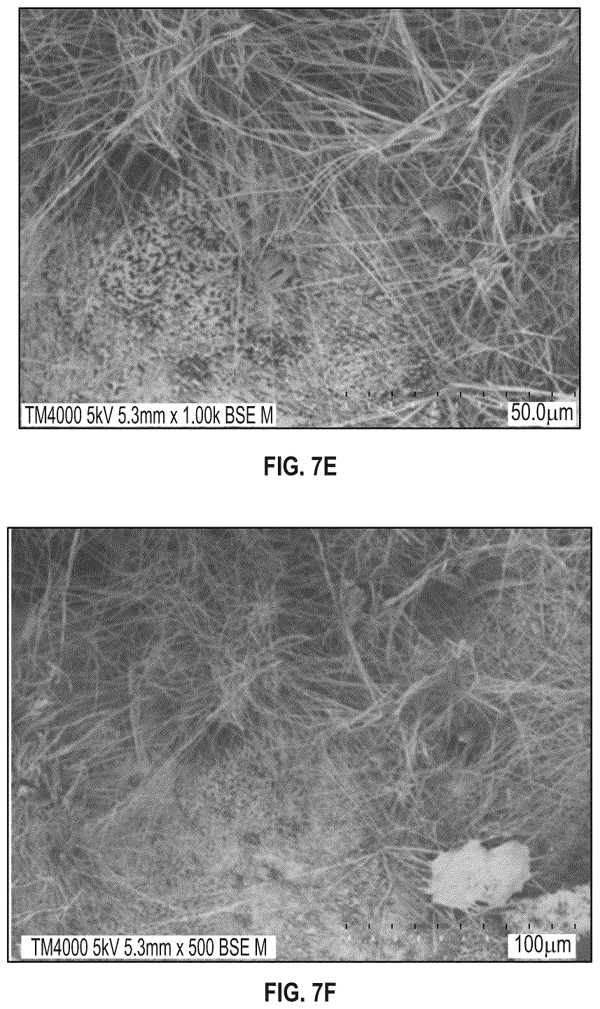

FIGS. 7A-7D are photographs illustrating the melt form process. Varying amounts of bupivacaine free base were placed in a mold (FIG. 7A) and heated to 100° C. for 3 minutes to melt the bupivacaine free base particles (FIG. 7B). The molten material was cooled for 1 minute (FIG. 7C) before being removed from the mold (FIG. 7D).

FIGS. 7E and 7F are scanning electron microscopy (SEM) images of depots formed using the melt form process at 500× (FIG. 7E) and 1000× (FIG. 7F) magnification. Depot samples were sputtered, and the backscattered electrons (BSE) signal was captured and used for imaging. The bupivacaine free base in the depot formed filament-like structures, which are hypothesized to provide structural integrity to the depot.

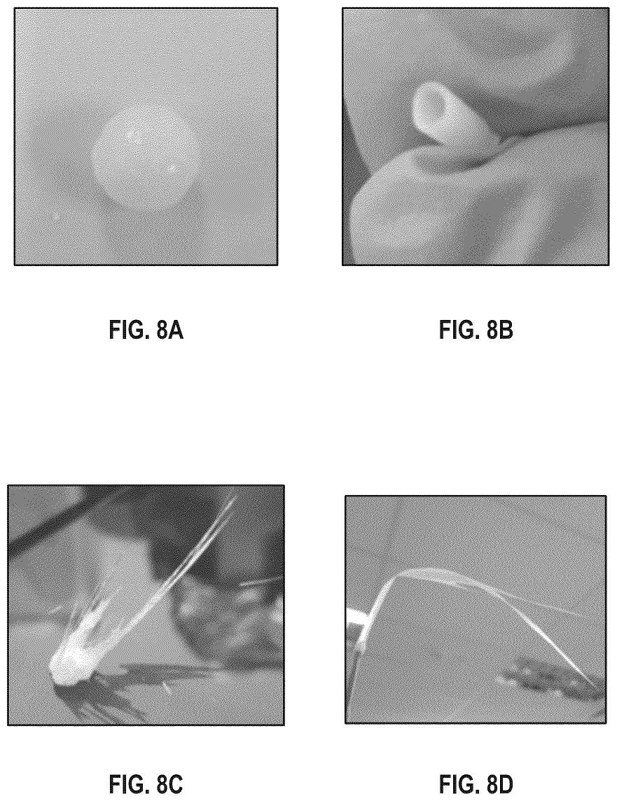

Depots formed using the above process were still malleable after removal from the mold, and thus could be manually remolded into various shapes. FIGS. 8A-8D are photographs illustrating example geometries for remolded, melt-formed implantable depots, including a sphere (FIG. 8A), a tube (FIG. 8B), filaments (FIG. 8C), and a thin film (FIG. 8D).

### Example 2: In Vitro Release from Bupivacaine Free Base Depots

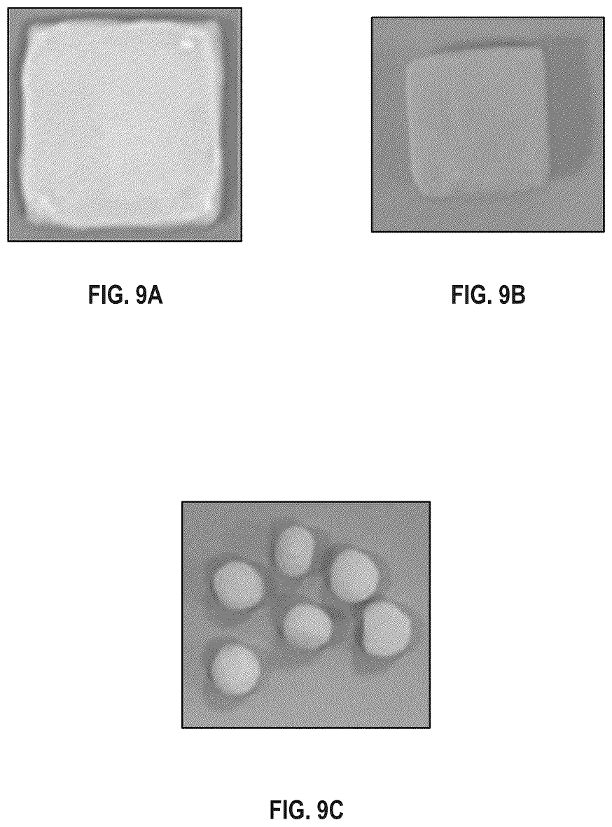

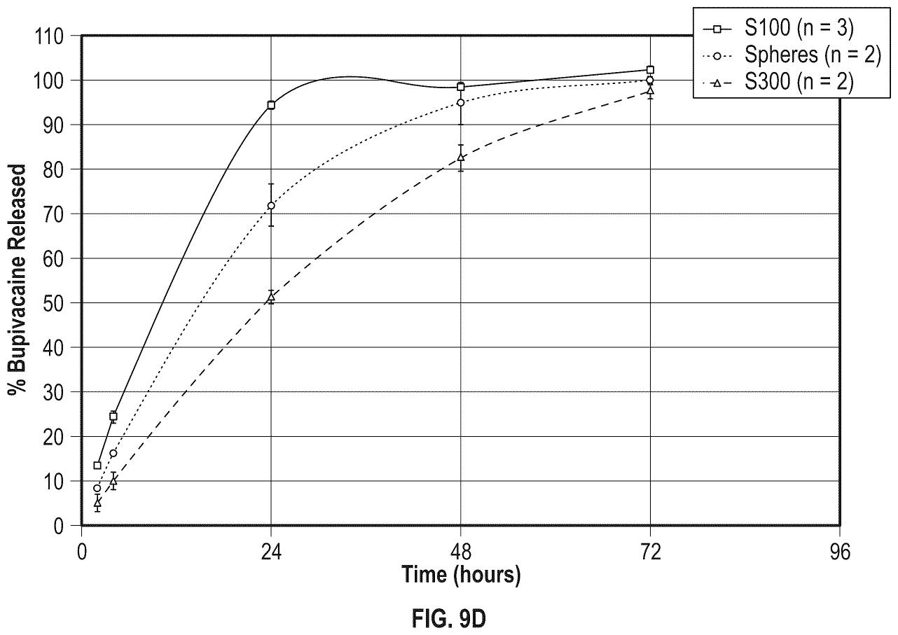

Three types of implantable depots were manufactured using the melt form process of Example 1. The compositions and geometry of the depots are listed in Table 1 below. FIGS. 9A-9C are photographs of the depots.

TABLE 1
Depot Compositions and Geometry

| Depot Type | Composition | Shape | Dimensions |
|---|---|---|---|
| S100 (FIG. 9A) | 100% bupivacaine free | Square | 10 mm × 10 mm × 1 |
| | base (100 mg) | | mm |
| S300 (FIG. 9B) | 100% bupivacaine free | Square | 10 mm × 10 mm × 3 |
| | base (300 mg) | | mm |
| Spheres (FIG. | 100% bupivacaine free | Spheres | 5 mm diameter |
| 9C) | base (300 mg, | | |
| | approximately 50 | | |
| | mg/sphere) | | |

FIG. 9D is a graph illustrating cumulative in vitro release of bupivacaine from the implantable depots. The release was obtained by using an accelerated in vitro release test in which the depots were immersed in a phosphate buffer at pH 5.8. At predetermined time points, aliquots of the buffer were drawn and analyzed using UV-Vis spectroscopy at 262 nm to quantify the amount of bupivacaine released. As shown in FIG. 9D, each of the three depots exhibited controlled release of their entire payload within 72 hours.

### Example 3: In Vivo Release from Bupivacaine Free Base Depots

An implantable depot was manufactured using the melt form process of Example 1. The composition and geometry of the depot is listed in Table 2 below.

TABLE 2
Depot Composition and Geometry

| Depot Type | Composition | Shape | Dimensions |
|---|---|---|---|
| S200 | 100% bupivacaine free | Square | 10 mm × 10 mm × 2 mm |
| | base (200 mg) | | |

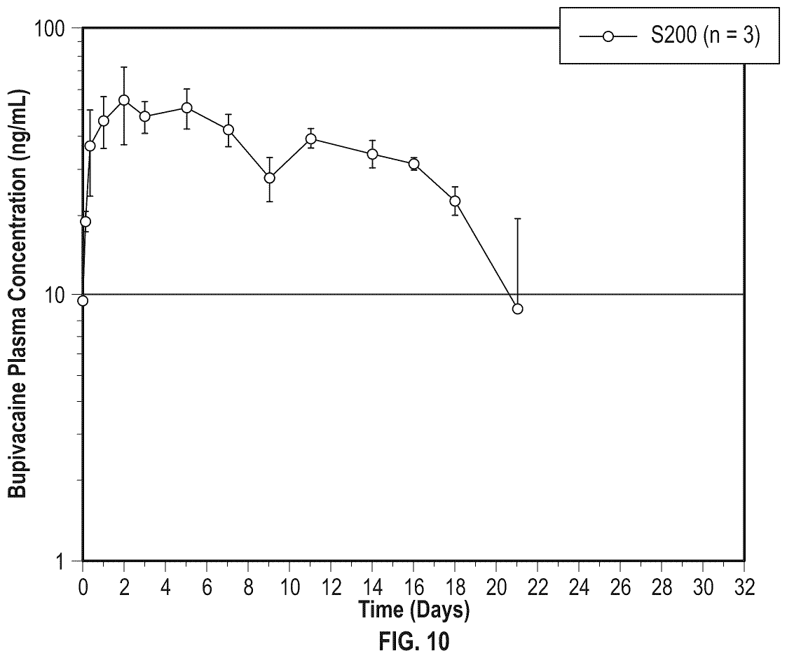

FIG. 10 is a semilog graph illustrating in vivo release of bupivacaine from the implantable depot in a rabbit subcutaneous model. 3 rabbits were each implanted with a single S200 depot in the subcutaneous space along the dorsal region. Only one subcutaneous pocket was created for the S200 depot. Blood draws were performed at predetermined time points (baseline, 1, 3, 8, 24, 48, 72, 120, 168, 216, 264, 336, 384, 432, 504, 600, and 672 hours). A bupivacaine assay was performed on each aliquot to quantify the plasma concentration of bupivacaine free base at each time point. As shown in FIG. 10, the S200 depots exhibited controlled release of bupivacaine free base for nearly 21 days.

### Example 4: Bupivacaine Free Base Coatings on an Implantable Mesh

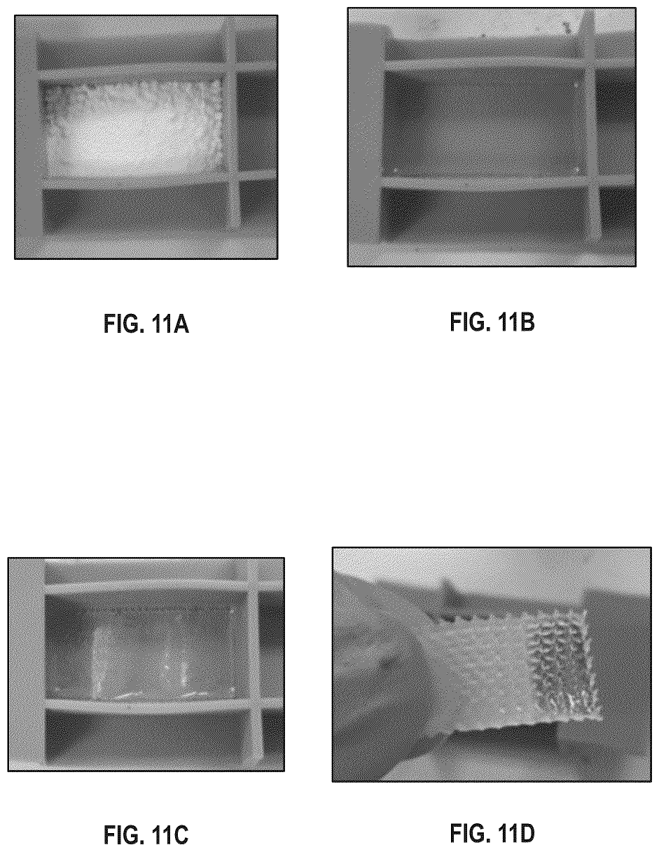

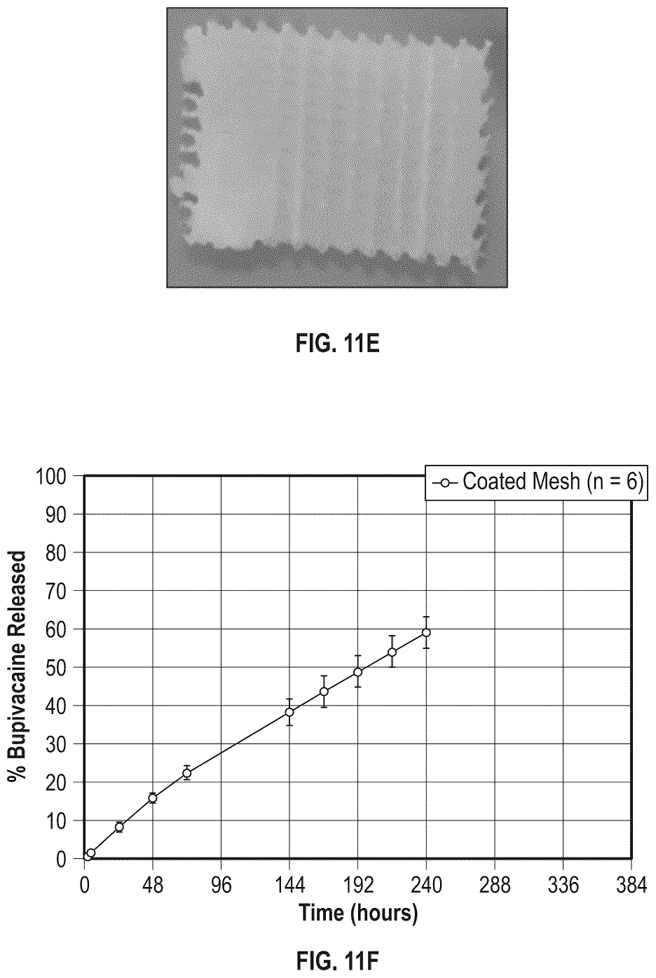

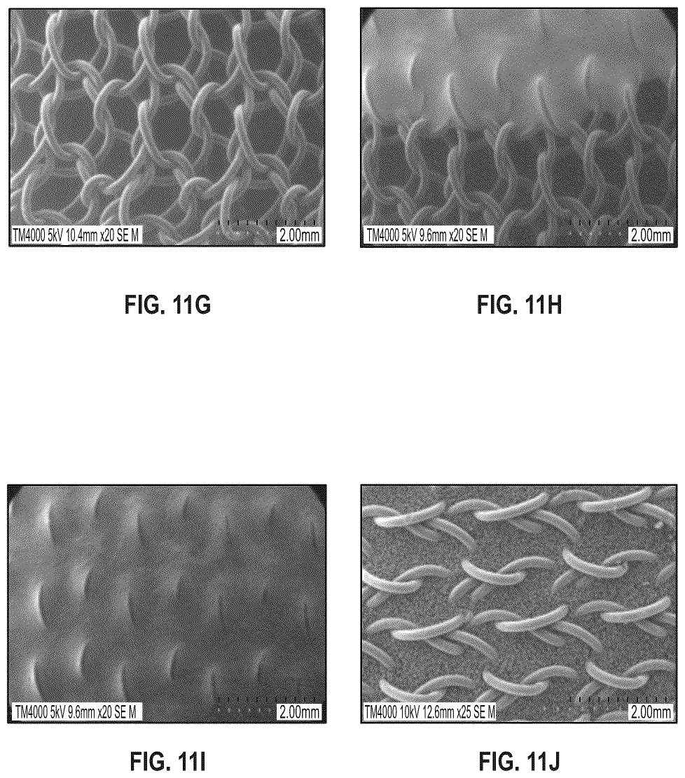

FIGS. 11A-11E are photographs illustrating a dip coating process for manufacturing implantable meshes coated with bupivacaine free base. The bupivacaine free base was heated to 100° C. for 3 minutes (FIGS. 11A and 11B). Once melted, a 20 mm×10 mm section of a hernia mesh (BARD® Mesh, monofilament polypropylene, 6 in×6 in) was dipped into the molten solution (FIG. 11C). After cooling, a coating of bupivacaine free base was formed on the mesh (FIGS. 11D and 11E). The total amount of bupivacaine free base coated onto each mesh was approximately 65 mg, for a dose density of 0.325 mg/mm². This weight in the coating was calculated by measuring the weight of the mesh before and after the coating was applied.

FIG. 11F is a graph illustrating cumulative in vitro release of bupivacaine from the coated mesh. The coated mesh was immersed in a phosphate buffer at pH 7.4. At predetermined time points, the coated mesh was removed from the pH 7.4 buffer and placed into fresh pH 7.4 buffer. The buffer was analyzed using UV-Vis spectroscopy to quantify the amount of bupivacaine free base released at each time point. As shown in FIG. 11F, the bupivacaine free base was released from the coated mesh in a controlled manner for more than a week. At t=10 days, approximately 60% of the total bupivacaine free base payload had eluted from the mesh.

FIGS. 11G and 11H are SEM images (20× magnification) showing the hernia mesh before (FIG. 11G) and after (FIG. 11H) the bupivacaine free base coating was applied. The mesh samples were sputtered, and the secondary electrons (SE) signal was captured and used for imaging.

FIGS. 111 and 11J are SEM images (20× magnification) showing the coated mesh at t=0 days, 0% release (FIG. 11I) and t=10 days, 60% release (FIG. 11J). As can be seen in FIG. 11J, the coating maintained its integrity even after the majority of the bupivacaine free base payload had eluted from the coating surface.

### Example 5: In Vitro Release from Bupivacaine Free Base Powder

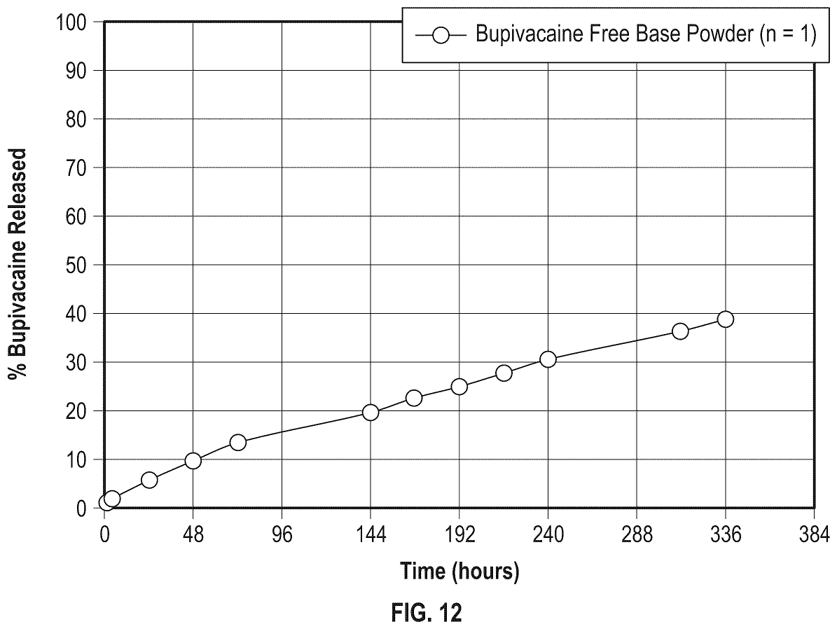

FIG. 12 is a graph illustrating cumulative in vitro release of bupivacaine from a powder. Bupivacaine free base was ground to an estimated particle size of 20 μm to 50 μm using a mortar and pestle. 100 mg of powder were immersed in a phosphate buffer at pH 7.4. At predetermined time points, the powder was removed from the pH 7.4 buffer and placed into fresh pH 7.4 buffer. The buffer was analyzed using UV-Vis spectroscopy to quantify the amount of bupivacaine free base released at each time point. As shown in FIG. 12, the bupivacaine was released from the powder in a controlled manner for more than two weeks. At t=14 days (336 hours), approximately 40% of the total bupivacaine free base payload had eluted from the powder.

### Example 6: Effect of Bupivacaine Free Base Particle Size on Release Rate

Bupivacaine free base powder with different particle sizes was prepared by grinding bupivacaine free base with a mortar and pestle, and then using a sieve to separate out smaller particles. Three different samples were prepared: (1) particles having an average diameter of approximately 172 μm (“200 μm particles”), (2) a mixture of particles with diameters ranging from 200 μm to 500 μm and having an average diameter of approximately 553 μm (“200-500 μm particle mixture”), and (3) particles having an average diameter of approximately 1483 μm (“1500 particles”).

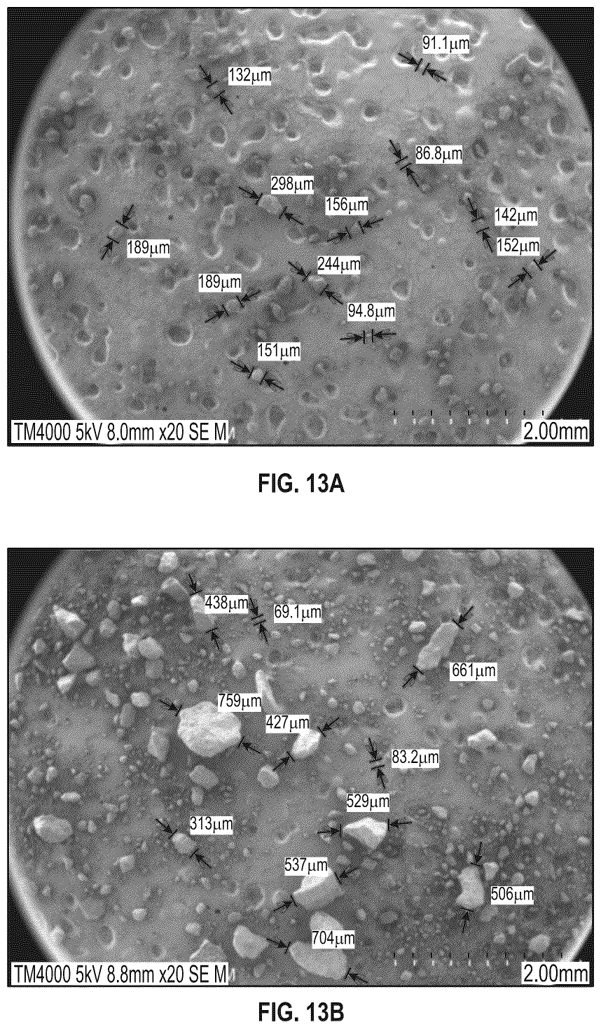

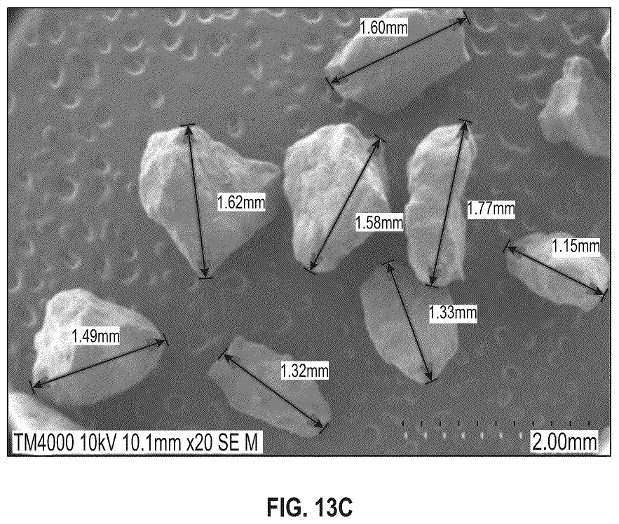

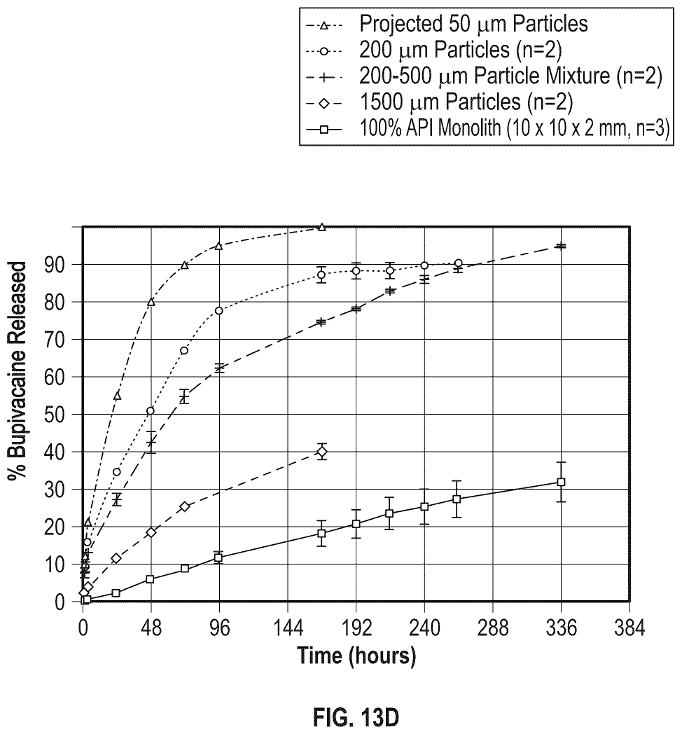

FIGS. 13A-13C are SEM images of the 200 μm particles (FIG. 13A), the 200-500 μm particle mixture (FIG. 13B), and the 1500 μm particles (FIG. 13C). The particle samples were sputtered, and the SE signal was captured and used for imaging. Particle size measurements were obtained by using the Hitachi TM4000 imaging software on the SEM images to measure the diameter of each particle; the measurements were averaged to determine the average size of each sample.

FIG. 13D is a graph illustrating cumulative in vitro release of bupivacaine from samples with varying form factors. The curves for the 200 μm particles, the 200-500 μm particle mixture, and the 1500 μm particles were obtained from in vitro release testing performed in phosphate buffer at pH 7.4 according to the protocol described in Example 5. The curve for the “projected 50 μm particles” was generated by determining the increase in release rate when reducing the particle size from 1500 μm to 200 μm, and then extrapolating the release rate for a 50 μm particle size. Specifically, the release rate is expected to correlate to the surface area to volume (SA/V) ratio of the particle. The SA/V ratio of a 1500 μm particle is 0.004, while the SA/V ratio of a 200 μm particle is 0.03, a 7.5-fold increase. When the particle size is reduced to 50 μm, the SA/V ratio increases to 0.12, which is a 4-fold increase over the 200 μm particles. Hence, the projected release rate for the 50 μm particles was expected be faster than the release rate of the 200 μm particles, but the difference in release rate between the 200 μm particles and the 50 μm particles is expected to be less than the difference in release rate between the 1500 μm particles and the 200 μm particles.

The curve for the “100% API monolith” was obtained from in vitro testing performed in phosphate buffer at pH 7.4 of a square depot (10 mm×10 mm×2 mm, containing 200 mg bupivacaine free base) fabricated using the melt form process of Example 1. As shown in FIG. 13D, for the bupivacaine free base powder, the release rate increased with decreasing particle size; smaller-sized particles had a faster release rate at each time point. Additionally, the monolithic square depot exhibited a much slower release rate compared to the free-flowing powder.

### ADDITIONAL EXAMPLES

Various examples of aspects of the present technology are described as numbered clauses (1, 2, 3, etc.) for convenience. These are provided as examples and do not limit the present technology.
Clause 1. An implantable depot for treating pain, the implantable depot comprising:
an analgesic constituting at least 50% of a total mass of the implantable depot, wherein at least some of the analgesic is in a free base form, and wherein, when implanted at a treatment site in vivo, the implantable depot is configured to release the analgesic over a release period of at least 3 days.
Clause 2. The implantable depot of Clause 1, wherein the implantable depot does not include any carrier materials for the analgesic.
Clause 3. The implantable depot of Clause 1 or 2, wherein the analgesic is not encapsulated by another material.
Clause 4. The implantable depot of any one of Clauses 1 to 3, wherein the analgesic is not dissolved in another material.
Clause 5. The implantable depot of any one of Clauses 1 to 4, wherein the analgesic constitutes at least 55%, 60%, 65%, 70%, 75%, 80%, 85%, 90%, 95%, 99%, or 100% of the total mass of the implantable depot.
Clause 6. The implantable depot of any one of Clauses 1 to 5, wherein the implantable depot comprises at least 25 mg, 50 mg, 100 mg, 200 mg, 300 mg, 400 mg, 500 mg, 1000 mg, or 2000 mg of the analgesic.
Clause 7. The implantable depot of any one of Clauses 1 to 6, wherein the analgesic comprises bupivacaine and the free base form comprises bupivacaine free base.
Clause 8. The implantable depot of any one of Clauses 1 to 7, wherein the analgesic comprises ropivacaine and the free base form comprises ropivacaine free base.
Clause 9. The implantable depot of any one of Clauses 1 to 8, wherein at least 10%, 20%, 30%, 40%, 50%, 60%, 70%, 80%, 90%, 95%, or 99% of the analgesic is in the free base form.
Clause 10. The implantable depot of any one of Clauses 1 to 8, wherein 100% of the analgesic is in the free base form.
Clause 11. The implantable depot of any one of Clauses 1 to 9, wherein at least some of the analgesic is in a salt form.
Clause 12. The implantable depot of Clause 11, wherein the analgesic comprises bupivacaine and the salt form comprises bupivacaine hydrochloride or bupivacaine hydrochloride monohydrate.
Clause 13. The implantable depot of Clause 11 or 12, wherein no more than 90%, 80%, 70%, 60%, 50%, 40%, 30%, 20%, or 10% of the analgesic is in the salt form.
Clause 14. The implantable depot of any one of Clauses 1 to 13, further comprising at least one excipient.
Clause 15. The implantable depot of Clause 14, wherein the at least one excipient comprises a hydrophilic excipient.
Clause 16. The implantable depot of Clause 14 or 15, wherein, when implanted, the at least one excipient is configured to form pores in the analgesic.
Clause 17. The implantable depot of any one of Clauses 14 to 16, wherein the at least one excipient comprises one or more of the following: polysorbate, polyethylene glycol, polyvinylpyrrolidone, poly (lactide-co-glycolide), sodium chloride, or sucrose.
Clause 18. The implantable depot of any one of Clauses 15 to 17, wherein the at least one excipient comprises a hydrophobic excipient.
Clause 19. The implantable depot of any one of Clauses 1 to 18, wherein the release period is at least 3 days, 4 days, 5 days, 6 days, 7 days, 10 days, 14 days, 21 days, 28 days, 30 days, 40 days, 50 days, 60 days, 70 days, 80 days, 90 days, 100 days, 110 days, or 120 days.
Clause 20. The implantable depot of any one of Clauses 1 to 19, wherein, when implanted at the treatment site in vivo, the implantable depot is configured to produce a mean blood plasma concentration of the analgesic of at least 10 ng/mL over the release period.
Clause 21. The implantable depot of any one of Clauses 1 to 20, wherein the implantable depot is a solid monolith.
Clause 22. The implantable depot of any one of Clauses 1 to 21, wherein the implantable depot is moldable.
Clause 23. The implantable depot of any one of Clauses 1 to 20, wherein the implantable depot comprises a plurality of particles.
Clause 24. The implantable depot of Clause 23, wherein the plurality of particles have an average diameter within a range from 10 μm to 100 μm.
Clause 25. The implantable depot of any one of Clauses 1 to 20, wherein the implantable depot comprises a coating on a medical device.
Clause 26. The implantable depot of Clause 25, wherein the medical device comprises a mesh, a prosthesis, an orthopedic implant, an antimicrobial implant, a film, a catheter, a pacemaker, a stent, a wound dressing, a wound closure device, an ear device, or a nasal splint.
Clause 27. A method for treating pain, the method comprising:
implanting a depot at a treatment site in vivo, wherein the depot comprises an analgesic constituting at least 50% of a total mass of the depot, and wherein at least some of the analgesic is in a free base form; and
releasing the analgesic over a release period of at least 3 days.
Clause 28. The method of Clause 27, wherein the pain comprises postoperative pain associated with a surgical procedure.
Clause 29. The method of Clause 28, wherein the surgical procedure comprises a knee surgery, a hip surgery, a shoulder surgery, a hernia repair surgery, a bunionectomy, a breast surgery, an abdominal surgery, a spine surgery, or a hemorrhoidectomy.
Clause 30. The method of Clause 27, wherein the pain comprises chronic pain.
Clause 31. The method of Clause 27, wherein the pain comprises pain resulting from a disease or condition.
Clause 32. The method of Clause 27, wherein the pain comprises pain resulting from a wound.
Clause 33. The method of any one of Clauses 27 to 32, wherein the depot does not include any carrier materials for the analgesic.
Clause 34. The method of any one of Clauses 27 to 33, wherein the analgesic is not encapsulated by another material.
Clause 35. The method of any one of Clauses 27 to 34, wherein the analgesic is not dissolved in another material.
Clause 36. The method of any one of Clauses 27 to 35, wherein the analgesic constitutes at least 55%, 60%, 65%, 70%, 75%, 80%, 85%, 90%, 95%, 99%, or 100% of the total mass of the depot.
Clause 37. The method of any one of Clauses 27 to 36, wherein the depot comprises at least 25 mg, 50 mg, 100 mg, 200 mg, 300 mg, 400 mg, 500 mg, 1000 mg, or 2000 mg of the analgesic.
Clause 38. The method of any one of Clauses 27 to 37, wherein the analgesic comprises bupivacaine and the free base form comprises bupivacaine free base.
Clause 39. The method of any one of Clauses 27 to 38, wherein the analgesic comprises ropivacaine and the free base form comprises ropivacaine free base.
Clause 40. The method of any one of Clauses 27 to 39, wherein at least 10%, 20%, 30%, 40%, 50%, 60%, 70%, 80%, 90%, 95%, or 99% of the analgesic is in the free base form.
Clause 41. The method of any one of Clauses 27 to 39, wherein 100% of the analgesic is in the free base form.
Clause 42. The method of any one of Clauses 27 to 40, wherein at least some of the analgesic is in a salt form.
Clause 43. The method of Clause 42, wherein the analgesic comprises bupivacaine and the salt form comprises bupivacaine hydrochloride or bupivacaine hydrochloride monohydrate.
Clause 44. The method of Clause 42 or 43, wherein no more than 90%, 80%, 70%, 60%, 50%, 40%, 30%, 20%, or 10% of the analgesic is in the salt form.
Clause 45. The method of any one of Clauses 27 to 44, wherein the depot comprises at least one excipient.
Clause 46. The method of Clause 45, wherein the at least one excipient comprises a hydrophilic excipient.
Clause 47. The method of Clause 45 or 46, wherein the at least one excipient comprises one or more of the following: polysorbate, polyethylene glycol, polyvinylpyrrolidone, poly (lactide-co-glycolide), sodium chloride, or sucrose.
Clause 48. The method of any one of Clauses 45 to 47, further comprising forming pores in the analgesic via the at least one excipient.
Clause 49. The method of any one of Clauses 45 to 48, wherein the at least one excipient comprises a hydrophobic excipient.
Clause 50. The method of any one of Clauses 27 to 49, wherein the release period is at least 3 days, 4 days, 5 days, 6 days, 7 days, 10 days, 14 days, 21 days, 28 days, 30 days, 40 days, 50 days, 60 days, 70 days, 80 days, 90 days, 100 days, 110 days, or 120 days.
Clause 51. The method of any one of Clauses 27 to 50, further comprising producing a mean blood plasma concentration of the analgesic of at least 10 ng/mL over the release period.
Clause 52. The method of any one of Clauses 27 to 51, wherein the depot comprises a solid monolith, a moldable material, a plurality of particles, a plurality of filaments, or a coating on a medical device.

### CONCLUSION

Although many of the embodiments are described above with respect to systems, devices, and methods for treating pain, the technology is applicable to other applications and/or other approaches, such as treating other diseases or conditions. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-13D.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded.

It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1.-52. (canceled)

53. An implantable depot for treating pain, the implantable depot comprising:
an analgesic constituting at least 50% of a total mass of the implantable depot, wherein at least some of the analgesic is in a free base form, and wherein, when implanted at a treatment site in vivo, the implantable depot is configured to release the analgesic over a release period of at least 3 days.

54. The implantable depot of claim 53, wherein the implantable depot does not include any carrier materials for the analgesic.

55. The implantable depot of claim 53, wherein the analgesic is not encapsulated by another material and is not dissolved in another material.

56. The implantable depot of claim 53, wherein the analgesic constitutes at least 90% of the total mass of the implantable depot.

57. The implantable depot of claim 53, wherein the analgesic comprises bupivacaine and the free base form comprises bupivacaine free base.

58. The implantable depot of claim 53, wherein the analgesic comprises ropivacaine and the free base form comprises ropivacaine free base.

59. The implantable depot of claim 53, wherein at least 50% of the analgesic is in the free base form.

60. The implantable depot of claim 59, wherein 100% of the analgesic is in the free base form.

61. The implantable depot of claim 53, wherein at least some of the analgesic is in a salt form.

62. The implantable depot of claim 61, wherein the analgesic comprises bupivacaine and the salt form comprises bupivacaine hydrochloride or bupivacaine hydrochloride monohydrate.

63. The implantable depot of claim 61, wherein no more than 50% of the analgesic is in the salt form.

64. The implantable depot of claim 53, further comprising at least one excipient.

65. The implantable depot of claim 64, wherein the at least one excipient comprises a hydrophilic excipient.

66. The implantable depot of claim 64, wherein the at least one excipient comprises one or more of the following: polysorbate, polyethylene glycol, polyvinylpyrrolidone, poly (lactide-co-glycolide), sodium chloride, or sucrose.

67. The implantable depot of claim 64, wherein the at least one excipient comprises a hydrophobic excipient.

68. The implantable depot of claim 53, wherein the release period is at least 7 days.

69. The implantable depot of claim 53, wherein the implantable depot is a solid monolith.

70. The implantable depot of claim 53 wherein the implantable depot is moldable.

71. The implantable depot of claim 53, wherein the implantable depot comprises a plurality of particles.

72. The implantable depot of claim 53, wherein the implantable depot comprises a coating on a medical device.
